(19) United States
(12) Patent Application Publication
(10) Pub. No.: US 2025/0188127 A1
(43) Pub. Date: Jun. 12, 2025

(54) BIOLOGICALLY PRODUCED NUCLEIC ACID FOR VACCINE PRODUCTION

(71) Applicant: Universität Basel, Basel (CH)

(72) Inventors: Enja Tatjana KIPFER, Liestal (CH); Thomas KLIMKAIT, Lörrach (DE); Christian MITTELHOLZER, Binningen (CH); Fabian OTTE, Efringen-Kirchen (DE)

(73) Assignee: Universität Basel, Basel (CH)

(21) Appl. No.: 18/690,452

(22) PCT Filed: Sep. 9, 2022
(86) PCT No.: PCT/EP2022/075140

§ 371 Date: Mar. 8, 2024

(30) Foreign Application Priority Data
Sep. 9, 2021 (EP) 21195643.8
Jun. 19, 2022 (EP) 22179777.2

(51) Int. Cl. C07K 14/005; A61K 39/215

(52) U.S. Cl. CPC C07K 14/005; A61K 39/215; C12N 2770/20022; C12N 2770/20034; C12N 2770/20051

(57) ABSTRACT

The invention relates to a biologically produced nucleic acid sequence comprising two or three primary nucleic acid sequence parts of SARS-COV-2 and not more than three secondary nucleic acid sequence parts, wherein a secondary nucleic acid sequence part encodes an amino acid sequence having the function of a SARS-COV-2 amino acid sequence encoded by ORF3a, ORF6, ORF7a or ORF8. The invention further relates to a host cell or a kit for producing the nucleic acid of the invention, a vector encoding the nucleic acid of the invention and products that can be obtained by the expression of the nucleic acid of the invention such as virus envelopes. The invention further relates a pharmaceutical composition comprising the nucleic acid of the invention or products derived thereof, preferably for use in the prevention of SARS-COV-2.

The invention relates to a biologically produced nucleic acid sequence comprising two or three primary nucleic acid sequence parts of SARS-COV-2 and not more than three secondary nucleic acid sequence parts, wherein a secondary nucleic acid sequence part encodes an amino acid sequence having the function of a SARS-COV-2 amino acid sequence encoded by ORF3a, ORF6, ORF7a or ORF8. The invention further relates to a host cell or a kit for producing the nucleic acid of the invention, a vector encoding the nucleic acid of the invention and products that can be obtained by the expression of the nucleic acid of the invention such as virus envelopes. The invention further relates a pharmaceutical composition comprising the nucleic acid of the invention or products derived thereof, preferably for use in the prevention of SARS-CoV-2.

The rapid development and availability of vaccines is crucial in combating many viruses and bacteria. The production of suitable vaccines is a multi-stage, complex process and is not always successful despite often high investments. Typically, the development of a suitable vaccine takes years. These long development times consist of a major problem, especially with regard to new emerging pathogens, or mutated pathogens, as from an epidemiological point of view it is only possible to react too late, if at all, to the emergence of new diseases. In contrast, the analysis, identification and further detection of new or heavily mutated pathogens are now possible within weeks or even days, which is a huge improvement over the last century.

In this context, viruses are of special interest, as they harbor high mutation rates causing the spread from other species to humans. Rapid spreading of these viruses makes them a major challenge for modern medicine. The usual time between the detection/identification of a newly emerging virus and the development of a vaccine is typically years. In a few cases, with sufficient prior knowledge, experimental vaccines could be provided within months. However, this time span is much longer than the typical time until thousands or millions of people are infected. Such rapid spread is also a direct consequence of the high mobility of today's society.

Ideally, immediately after the identification of a new virus, a vaccine would be available in sufficient quantity and of the highest quality and would allow for a nationwide vaccination of all persons who have somehow come close to the initial outbreak site of the new virus. Furthermore, an ideal method for such a vaccine would be capable of reacting to the evolution and adaptation of the virus. Such an ideal production possibility seems utopian to the person skilled in the art today.

In the recent past in particular, the corona pandemic has dramatically increased the relevance of developing suitable tools for vaccine production. There is unanimous agreement that the development of a vaccine against the coronavirus SARS-COV-2 is the only proven means of containing the pandemic and the associated global crisis in the long term.

Thus, there is a need for to provide an instrument which allows the production of a vaccine against the coronavirus SARS-COV-2, in large quantities and of high quality.

The above technical problem is solved by the embodiments disclosed herein and as defined in the claims.

Accordingly, the invention relates to, inter alia, the following embodiments:
1. A biologically produced nucleic acid sequence comprising
   a) two or three primary nucleic acid sequence parts, wherein a primary nucleic acid sequence part encodes an amino acid sequence selected from the group consisting of
      i) SEQ ID NO: 1 (SARS-COV-2 N) or an amino acid sequence with at least 90% sequence identity thereof;
      ii) SEQ ID NO: 2 (SARS-COV-2 S) or an amino acid sequence with at least 90% sequence identity thereof;
      iii) SEQ ID NO: 3 (SARS-COV-2 E) or an amino acid sequence with at least 90% sequence identity thereof; and
      iv) SEQ ID NO: 4 (SARS-COV-2 M) or an amino acid sequence with at least 90% sequence identity thereof; and
   b) not more than three, not more than two not more than one or no secondary nucleic acid sequence part(s), wherein a secondary nucleic acid sequence part encodes an amino acid sequence having the function of a SARS-COV-2 amino acid sequence encoded by ORF3a, ORF6, ORF7a, or ORF8, wherein if no sequence part of a) iii) and no nucleic acid sequence part that encodes an amino acid sequence having the function of a SARS-COV-2 amino acid sequence encoded by ORF3a are present, then not more than five, not more than four, not more than three nucleic acid sequence parts selected from a) i), a) ii), a) iv), and nucleic acid sequence parts encoding an amino acid sequence having the function of a SARS-COV-2 amino acid sequence encoded by ORF6, ORF7a, or ORF8 are present.
2. The nucleic acid sequence of embodiment 1, wherein the nucleic acid sequence comprises two or three primary nucleic acid sequence parts, wherein a primary nucleic acid sequence part encodes an amino acid sequence selected from the group consisting of
   i) SEQ ID NO: 1 (SARS-COV-2 N) or an amino acid sequence with at least 90% sequence identity thereof;
   ii) SEQ ID NO: 2 (SARS-COV-2 S) or an amino acid sequence with at least 90% sequence identity thereof; and
   iii) SEQ ID NO: 3 (SARS-COV-2 E) or an amino acid sequence with at least 90% sequence identity thereof; and
      wherein the nucleic acid sequence has no sequence part that encodes an amino acid sequence having the function of a SARS-COV-2 amino acid sequence encoded by SEQ ID NO: 4 (SARS-COV-2 M).
3. The nucleic acid sequence of embodiment 2, wherein the nucleic acid sequence comprises three primary nucleic acid sequence parts:
   i) SEQ ID NO: 1 (SARS-COV-2 N) or an amino acid sequence with at least 90% sequence identity thereof;
   ii) SEQ ID NO: 2 (SARS-COV-2 S) or an amino acid sequence with at least 90% sequence identity thereof; and
   iii) SEQ ID NO: 3 (SARS-COV-2 E) or an amino acid sequence with at least 90% sequence identity thereof.
4. The nucleic acid sequence of embodiment 2 or 3, wherein
   1.) the nucleic acid sequence comprises no nucleic acid sequence part encodes an amino acid sequence having the function of a SARS-COV-2 amino acid sequence encoded by ORF7 and ORF8;
   2.) the nucleic acid sequence comprises no nucleic acid sequence part encodes an amino acid sequence having the function of a SARS-COV-2 amino acid sequence encoded by ORF6 and ORF7ab; or
   3.) the nucleic acid sequence comprises no nucleic acid sequence part encodes an amino acid sequence having the function of a SARS-COV-2 amino acid sequence encoded by ORF6, ORF7ab and ORF8.
5. The nucleic acid sequence of embodiment 4, wherein the nucleic acid sequence comprises a primary nucleic acid sequence part encoding an amino acid sequence a) i), a secondary nucleic acid sequence part encoding an amino acid sequence having the function of a SARS-COV-2 amino acid sequence encoded by ORF3a and a sequence part of the nucleic acid sequence located between the primary nucleic acid sequence part encoding an amino acid sequence a) i) and the secondary nucleic acid sequence part encoding an amino acid sequence having the function of a SARS-COV-2 amino acid sequence encoded by ORF3a, wherein the sequence part comprises
   I) SEQ ID NO: 35 or a sequence having at least 90% sequence identity to SEQ ID NO: 35;
   II) SEQ ID NO: 36 or a sequence having at least 90% sequence identity to SEQ ID NO: 36; or
   III) SEQ ID NO:37 or a sequence having at least 90% sequence identity to SEQ ID NO: 37.
6. A biologically produced nucleic acid sequence comprising two or three nucleic acid sequence parts encoding an amino acid sequence selected from the group consisting of:
   i) SEQ ID NO: 1 (SARS-COV-2 N) or an amino acid sequence with at least 90% sequence identity thereof;
   ii) SEQ ID NO: 2 (SARS-COV-2 S) or an amino acid sequence with at least 90% sequence identity thereof; and
   iii) SEQ ID NO: 3 (SARS-COV-2 E) or an amino acid sequence with at least 90% sequence identity thereof; and
      wherein the nucleic acid sequence has no sequence part that encodes an amino acid sequence having the function of a SARS-COV-2 amino acid sequence encoded by SEQ ID NO: 4 (SARS-COV-2 M),
      preferably wherein the nucleic acid sequence comprises a sequence as defined by SEQ ID NO: 33.
7. The nucleic acid sequence of embodiment 3, wherein the nucleic acid sequence comprises two primary nucleic acid sequence parts:
   i) SEQ ID NO: 1 (SARS-COV-2 N) or an amino acid sequence with at least 90% sequence identity thereof; and
   ii) SEQ ID NO: 2 (SARS-COV-2 S) or an amino acid sequence with at least 90% sequence identity thereof; and
      wherein the nucleic acid sequence has no sequence part that encodes an amino acid sequence having the function of a SARS-COV-2 amino acid sequence encoded by SEQ ID NO: 4 (SARS-COV-2 M) and SEQ ID NO: 3 (SARS-COV-2 E),
      preferably wherein the nucleic acid sequence comprises a sequence as defined by SEQ ID NO: 34.
8. The nucleic acid sequence of embodiment 1, wherein for the secondary nucleic acid sequence part encoding an amino acid sequence having the function of a SARS-COV-2 amino acid sequence
   i) ORF3a is a sequence defined by SEQ ID NO: 5;
   ii) ORF6 is a sequence defined by SEQ ID NO: 6;
   iii) ORF7a is a sequence defined by SEQ ID NO: 7; and/or
   iv) ORF8 is a sequence defined by SEQ ID NO: 9.
9. The nucleic acid sequence of embodiment 1 or 8, wherein the nucleic acid sequence comprises three primary nucleic acid sequence parts.
10. The nucleic acid sequence of any one of embodiments 1, 8 or 9, wherein one of the secondary nucleic acid sequence parts encodes an amino acid sequence having the function of a SARS-COV-2 amino acid sequence encoded by ORF3a.
11. The nucleic acid sequence of any one of embodiments 1, 8 to 10, wherein the primary nucleic acid sequence parts and the secondary nucleic acid sequence parts are ordered in 5′ to 3′ direction in the following order:
   1. SEQ ID NO: 2 (SARS-COV-2 S) or an amino acid sequence with at least 90% sequence identity thereof,
   2. nucleic acid sequence part encoding an amino acid sequence having the function of a SARS-COV-2 amino acid sequence encoded by ORF3a;
   3. SEQ ID NO: 3 (SARS-COV-2 E) or an amino acid sequence with at least 90% sequence identity thereof,
   4. SEQ ID NO: 4 (SARS-COV-2 M) or an amino acid sequence with at least 90% sequence identity thereof,
   5. nucleic acid sequence part encoding an amino acid sequence having the function of a SARS-COV-2 amino acid sequence encoded by ORF6,
   6. nucleic acid sequence part encoding an amino acid sequence having the function of a SARS-COV-2 amino acid sequence encoded by ORF7a,
   7. nucleic acid sequence part encoding an amino acid sequence encoding an amino acid sequence having the function of a SARS-COV-2 amino acid sequence encoded by ORF8,
   8. SEQ ID NO: 1 (SARS-COV-2 N) or an amino acid sequence with at least 90% sequence identity thereof.
12. The nucleic acid sequence of any one of embodiments 1, 8 to 11, wherein the nucleic acid sequence comprises a nucleic acid sequence defined by the SEQ ID NO: 10 (SARS-COV-2 genome) or a sequence with at least 90% sequence identity thereof with a deletion and/or a dysfunctionality of:
   a) the E gene, ORF6 gene, ORF7a gene and ORF8 gene; or
   b) the E gene, ORF6 gene and ORF8 gene.
13. A vector comprising the nucleic acid sequence of one of the preceding embodiments.
14. The vector of embodiment 13, wherein the vector is a plasmid vector.
15. The vector of embodiment 14, wherein the vector comprises
   a) a sequence as defined by SEQ ID NO: 11 (biologically produced vector with ORF7a gene) or a sequence having 90% sequence identity thereof; or
   b) a sequence as defined by SEQ ID NO: 12 (biologically produced vector without ORF7a gene) or a sequence having 90% sequence identity thereof.
16. A host cell comprising the nucleic acid sequence of embodiments 1 to 12 or the vector of embodiments 13 to 15.
17. The host cell of embodiment 16 additionally comprising at least one complementary SARS-COV-2 sequence thereof.
18. A method of production of a virus envelope and/or a fragment of a virus envelope and/or virus envelope protein comprising culturing the host cell of embodiment 16 or 16.
19. A kit comprising
   I.) the nucleic acid sequence of any one of the of embodiments 1 to 12, the vector of embodiments 13 to 15, or the host cell of embodiment 16; and
   II.) at least one SARS-COV-2 sequence part complementary to the nucleic acid sequence comprised in (I.).
20. A virus envelope or a fragment of a virus envelope and/or virus envelope protein, wherein the virus envelope or the fragment of a virus envelope and/or the virus envelope protein
   a) package the at least one nucleic acid of any one of embodiments 1 to 12; and
   b) are obtainable by gene expression using at least one nucleic acid of any one of embodiments 1 to 6, using the vector of any one of embodiments 13 to 15, using the host cell of embodiment 16 or 17, using the method of embodiment 18 or using the kit of embodiment 19.
21. A pharmaceutical composition comprising
   a) at least one nucleic acid according to one of embodiments 1 to 12; and
   b) at least one amino acid sequence obtainable by gene expression using at least one nucleic acid of any one of embodiments 1 to 12, using the vector of any one of embodiments 13 to 15, using the host cell of embodiment 16 or 17, using the method of embodiment 18 or using the kit of embodiment 19.

22. The pharmaceutical composition of embodiment 21, wherein the at least one amino acid sequence is the virus envelope or a fragment of a virus envelope and/or virus envelope protein of embodiment 20.

23. The pharmaceutical composition according to embodiment 21 or 22 for use as a medicament.

24. The pharmaceutical composition according to embodiment 21 or 22 for use in the prevention of a SARS-COV-2 infection or at least one symptom thereof.

Accordingly, the invention relates to a nucleic acid sequence comprising a) two or three primary nucleic acid sequence parts, wherein a primary nucleic acid sequence part encodes an amino acid sequence selected from the group consisting of i) SEQ ID NO: 1 (SARS-COV-2 N) or an amino acid sequence with at least 90%, at least 91%, at least 92%, at least 93%, at least 94%, at least 95%, at least 96%, at least 97%, at least 98%, or at least 99% sequence identity thereof; ii) SEQ ID NO: 2 (SARS-COV-2 S) or an amino acid sequence with at least 90%, at least 91%, at least 92%, at least 93%, at least 94%, at least 95%, at least 96%, at least 97%, at least 98%, or at least 99% sequence identity thereof; iii) SEQ ID NO: 3 (SARS-COV-2 E) or an amino acid sequence with at least 90%, at least 91%, at least 92%, at least 93%, at least 94%, at least 95%, at least 96%, at least 97%, at least 98%, or at least 99% sequence identity thereof; and iv) SEQ ID NO: 4 (SARS-COV-2 M) or an amino acid sequence with at least 90%, at least 91%, at least 92%, at least 93%. at least 94%, at least 95%, at least 96%, at least 97%, at least 98%, or at least 99% sequence identity thereof; and b) not more than three, not more than two not more than one or no secondary nucleic acid sequence part(s), secondary nucleic acid sequence parts, wherein each secondary nucleic acid sequence part encodes an amino acid sequence having the function of a SARS-COV-2 amino acid sequence encoded by ORF3a, ORF6, ORF7a, or ORF8, wherein if no sequence part of a) iii) and no nucleic acid sequence part that encodes an amino acid sequence having the function of a SARS-COV-2 amino acid sequence encoded by ORF3a are present, then not more than five, not more than four, not more than three nucleic acid sequence parts selected from a) i), a) ii), a) iv), and nucleic acid sequence parts encoding an amino acid sequence having the function of a SARS-COV-2 amino acid sequence encoded by ORF6, ORF7a, or ORF8 are present.

The nucleic acid of the invention is preferably biologically produced.

The term “nucleic acid sequence”, as used herein, refers to either DNA, RNA, and any modifications thereof. The nucleic acid may be single-stranded or double-stranded. Modifications include, but are not limited to, those which provide other chemical groups that incorporate additional charge, polarizability, hydrogen bonding, electrostatic interaction, and fluxionality to the nucleic acid ligand bases or the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine. Modifications can also include 3′ and 5′ modifications such as capping.

Any deoxyribonucleic acid described herein, may alternatively refer to a corresponding ribonucleic acid. In these, the corresponding ribonucleic acid has sequence parts as defined above in which thymine (T) is replaced by uracil (U).

The terms “primary” and “secondary”, as used herein, is used to distinguish between two groups of nucleic acid without necessarily describing a structural property.

The term “percent (%) sequence identity” with respect to a reference sequence is defined as the percentage of nucleotides or amino acid residues in a candidate sequence that are identical with the nucleotides or amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

In some embodiments, the nucleotide acid sequence of the invention is altered (e.g., to facilitate the production process of the nucleotide acid sequence or products thereof) without altering or by unsubstantially altering the properties of the protein products.

In some embodiments, the alterations of the nucleotide acid sequence of the invention include at least one alteration selected from the group of
1) base substitutions insertions, or deletions relative to the reference sequence without altering or by unsubstantially altering the properties of the protein products;
2) replacing codons with synonymous versions; and
3) reduction of the number of hypothetical genetic elements present within protein-coding sequences such as (alternative) ORFs, predicted gene internal transcriptional start sites, and/or sequence motifs (predicted or cryptic) that fine-tune translation rates (e.g., ribosome stalling motifs).

Testing whether the genes of the altered nucleotide acid sequence of the invention remain functional will identify genes in which additional information beyond the amino acid code is necessary for proper functioning.

In some embodiments, the nucleotide acid sequence described herein is altered to improve the biological function of the encoded protein products.

Such a biological function includes but is not limited to stability enhancement, production facilitation (e.g., insertion of additional replication initiating sequences), altered antigenicity, replication limitation.

In some embodiments, the nucleotide acid sequence described herein is altered to encode at least one alternative protein of interest with a similar structure but alternative biological function, such as the function of a protein of a SARS-COV-2 variant.

The person skilled in the art can obtain such an altered nucleotide sequence by analyzing the sequence coding for at least one alternative protein of interest (e.g. the nucleotide acid sequence of a mutated virus) and implementing the relevant alterations (e.g. mutations) into the corresponding nucleotide acid sequence described herein.

In some embodiments, the SARS-COV-2 described herein is a SARS-COV-2 variant comprising at least one mutation selected from the group of del 69-70, RSYLTPGD246-253N, N440K, G446V, L452R, Y453F, S477G/N, E484Q, E484K, F490S, N501Y, N501S, D614G, Q677P/H, P681H and P681R.

In some embodiments, the SARS-COV-2 described herein is a SARS-COV-2 variant selected from the group of Lineage B.1.1.207, Lineage B.1.1.7, Cluster 5, 501.V2 variant, Lineage P.1, Lineage B.1.429/CAL.20C, Lineage B.1.525, Lineage B1.620, Lineage C 37 and Lineage B.1.621.

In some embodiments, the SARS-COV-2 described herein is a SARS-COV-2 variant described by a Nextstrain clade selected from the group 19A, 20A, 20C, 20G, 20H, 20B, 20D, 20F, 20I, 20E and 21A.

The skilled person is aware of how to implement additional mutations or combination of mutations described herein based on new occurring SARS-COV-2 variants.

In some embodiments, the sequence coding for at least one alternative protein of interest comprises sequences coding for a protein that is characteristic for at least one SARS-COV-2 variant. In some embodiments, the protein that is characteristic for at least one SARS-COV-2 variant is a protein that is encoded by a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the sequences SEQ ID NO: 13, SEQ ID NO: 14, SEQ ID NO: 15 and/or SEQ ID NO: 16.

This implementation of the relevant alterations can be achieved for example by insertion, deletion, substitution, and/or modification of at least one base, but not more than the percentage of the nucleotide acid sequence described herein.

The term “biologically produced”, as used herein, means that oligomeric fragments of the nucleic acid according to the invention with a length of less than 1000 bases are produced by at least one PCR-driven molecular biology technique. Therefore, the short oligomers are not exclusively produced by chemical reaction steps using chemical reagents. PCR-driven molecular biology techniques may also be used during individual late production steps, such as the joining of already longer oligomers. The latter can in turn optionally be synthetic. Biologically produced nucleic acids can be identical to naturally occurring nucleic acids. Biologically produced nucleic acids differ from fully synthetic nucleic acids in one or more of the following sequence features:
1.) the presence of one or more enzymatic restriction sites, in particular, restriction sites for Type IIS restriction endonucleases, which are known to the person skilled in the art;
2.) the presence or increased occurrence, compared to the corresponding fully synthetic nucleic acids, of repeating nucleic acid sequences with more than 9 consecutive units of the same base within the biologically produced nucleic acid nucleic acid;
3.) the presence or increased occurrence of repeating base-pair sequences with more than 12 bases compared to the corresponding fully synthetic nucleic acids;
4.) the presence or increased occurrence, relative to the corresponding fully synthetic nucleic acids, of indirectly repeating base-pair segments consisting of more than 12 base units known to the person skilled in the art as reverse-complementary sequences therefor;
5.) the presence or increased occurrence, relative to the corresponding fully synthetic nucleic acids, of nucleic acid sequences with more than 9 consecutive repetitions of duplicate base units (dinucleotide repeats) known to the person skilled in the art; and
6.) the presence or increased occurrence, relative to the corresponding fully synthetic nucleic acids, of nucleic acid sequences with more than five consecutive repetitions of triple base units (trinucleotide repeats) known to the person skilled in the art.

The phrase “sequence having the function of a SARS-COV-2 amino acid sequence”, as used herein, refers to a sequence having the function of a SARS-COV-2 amino acid sequence encoded by the sequence as defined by the SEQ ID NO: 10. The structure and function of SARS-COV-2 amino acid sequences are known in the art (see e.g. Yadav, Rohitash et al., 2021, Cells vol. 10,4 821; Arya, Rimanshee, et al., 2021, Journal of molecular biology 433.2:166725; Gorkhali, R., et al., 2021, Bioinformatics and Biology Insights, 15, 11779322211025876; Redondo N, et al., 2021, Front Immunol. July 7;12:708264). In some embodiments, the sequence having the function of a SARS-COV-2 amino acid sequence described herein is a sequence comprised in SEQ ID NO: 10 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the sequence comprised in SEQ ID NO: 10. Such % sequence variation can for example derive from one or more mutations of a SARS-CoV-2 variant in the SEQ ID NO: 10 or from insertions, deletions and/or replacements, preferably conservative insertions, deletions and/or replacements that alter the sequence without altering or without substantially altering the function of the encoded amino acid sequence.

The function of a SARS-COV-2 amino acid sequence encoded by ORF3a as well as ORF3a sequences and mutations thereof are known in the art (see e.g. Bianchi M, et al., 2021, Int J Biol Macromol. 2021; 170:820-826. ) The most common mutations in the ORF3a sequence are V13L, Q57H, Q57H+A99V, G196V and G252V. In some embodiments, the secondary nucleic acid sequence part encoding an amino acid sequence having the function of ORF3a is a sequence encoding SEQ ID NO: 5 or a sequence encoding SEQ ID NO: 5 having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the sequence encoding SEQ ID NO: 5. In some embodiments, the secondary nucleic acid sequence part encoding an amino acid sequence having the function of ORF3a is a sequence as defined by SEQ ID NO: 17 having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 17. Such % sequence variation can for example derive from one or more mutations described in Bianchi M, et al., 2021, Int J Biol Macromol. 2021;170:820-826 or from insertions, deletions and/or replacements, preferably conservative insertions, deletions and/or replacements that alter the sequence without altering or without substantially altering the function of the encoded amino acid sequence.

The function of a SARS-COV-2 amino acid sequence encoded by ORF6 as well as ORF6 sequences and mutations thereof are known in the art (see e.g. Hassan, Sk Sarif, Pabitra Pal Choudhury, and Bidyut Roy, 2021, Meta Gene 28:100873.) In some embodiments, the secondary nucleic acid sequence part encoding an amino acid sequence having the function of ORF6 is a sequence encoding SEQ ID NO: 6 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the sequence encoding SEQ ID NO: 6. In some embodiments, the secondary nucleic acid sequence part encoding an amino acid sequence having the function of ORF6 is a sequence as defined by SEQ ID NO: 18 having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 18. Such % sequence variation can for example derive from one or more mutations described in Hassan, Sk Sarif, Pabitra Pal Choudhury, and Bidyut Roy, 2021, Meta Gene 28:100873 or from insertions, deletions and/or replacements, preferably conservative insertions, deletions and/or replacements that alter the sequence without altering or without substantially altering the function of the encoded amino acid sequence.

The function of a SARS-COV-2 amino acid sequence encoded by ORF7a as well as ORF7a sequences and mutations thereof are known in the art (see e.g. Yashvardhini, Niti, et al., 2021, Biomedical Research and Therapy 8.8:4497-4504.) In some embodiments, the secondary nucleic acid sequence part encoding an amino acid sequence having the function of ORF7a is a sequence encoding SEQ ID NO: 7 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the sequence encoding SEQ ID NO: 7. In some embodiments, the secondary nucleic acid sequence part encoding an amino acid sequence having the function of ORF7a is a sequence as defined by SEQ ID NO: 19 having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 19. Such % sequence variation can for example derive from one or more mutations described in Yashvardhini, Niti, et al., 2021, Biomedical Research and Therapy 8.8:4497-4504 or from insertions, deletions and/or replacements, preferably conservative insertions, deletions and/or replacements that alter the sequence without altering or without substantially altering the function of the encoded amino acid sequence.

The function of a SARS-COV-2 amino acid sequence encoded by ORF7b as well as ORF7b sequences and mutations thereof are known in the art (see e.g. Hassan, Sk Sarif, Pabitra Pal Choudhury, and Bidyut Roy, 2021, Meta Gene 28:100873.) In some embodiments, the secondary nucleic acid sequence part encoding an amino acid sequence having the function of ORF7b is a sequence encoding SEQ ID NO: 8 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the sequence encoding SEQ ID NO: 8. In some embodiments, the secondary nucleic acid sequence part encoding an amino acid sequence having the function of ORF7b is a sequence as defined by SEQ ID NO: 20 having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 20. Such % sequence variation can for example derive from one or more mutations described in Hassan, Sk Sarif, Pabitra Pal Choudhury, and Bidyut Roy, 2021, Meta Gene 28:100873 or from insertions, deletions and/or replacements, preferably conservative insertions, deletions and/or replacements that alter the sequence without altering or without substantially altering the function of the encoded amino acid sequence.

The function of a SARS-COV-2 amino acid sequence encoded by ORF8 as well as ORF8 sequences and mutations thereof are known in the art (see e.g. Badua, Christian Luke DC, Karol Ann T. Baldo, and Paul Mark B. Medina., 2021, Journal of medical virology 93.3:1702-1721; Hassan, Sk Sarif, et al., 2021, Computers in biology and medicine 133:104380.) In some embodiments, the secondary nucleic acid sequence part encoding an amino acid sequence having the function of ORF8 is a sequence encoding SEQ ID NO: 9 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the sequence encoding SEQ ID NO: 9. In some embodiments, the secondary nucleic acid sequence part encoding an amino acid sequence having the function of ORF8 is a sequence as defined by SEQ ID NO: 21 having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 21. Such % sequence variation can for example derive from one or more mutations described in Badua, Christian Luke DC, Karol Ann T. Baldo, and Paul Mark B. Medina., 2021, Journal of medical virology 93.3:1702-1721 or from insertions, deletions and/or replacements, preferably conservative insertions, deletions and/or replacements that alter the sequence without altering or without substantially altering the function of the encoded amino acid sequence.

In some embodiments, the invention relates to a nucleic acid sequence described herein, wherein a) comprises two primary nucleic acid sequence parts, wherein one primary nucleic acid sequence part encodes SEQ ID NO: 1 (SARS-COV-2 N) or an amino acid sequence with at least 90%, at least 91%, at least 92%, at least 93%, at least 94%, at least 95%, at least 96%, at least 97%, at least 98%, or at least 99% sequence identity thereof; and wherein one primary nucleic acid sequence part encodes SEQ ID NO: 2 (SARS-COV-2 S) or an amino acid sequence with at least 90%, at least 91%, at least 92%, at least 93%, at least 94%, at least 95%, at least 96%, at least 97%, at least 98%, or at least 99% sequence identity thereof.

In some embodiments, the invention relates to a nucleic acid sequence described herein, wherein a) comprises two primary nucleic acid sequence parts, wherein one primary nucleic acid sequence part encodes SEQ ID NO: 1 (SARS-COV-2 N) or an amino acid sequence with at least 90%, at least 91%, at least 92%, at least 93%, at least 94%, at least 95%, at least 96%, at least 97%, at least 98%, or at least 99% sequence identity thereof; and wherein one primary nucleic acid sequence part encodes SEQ ID NO: 3 (SARS-COV-2 E) or an amino acid sequence with at least 90%, at least 91%, at least 92%, at least 93%, at least 94%, at least 95%, at least 96%, at least 97%, at least 98%, or at least 99% sequence identity thereof.

In some embodiments, the invention relates to a nucleic acid sequence described herein, wherein a) comprises two primary nucleic acid sequence parts, wherein one primary nucleic acid sequence part encodes SEQ ID NO: 1 (SARS-COV-2 N) or an amino acid sequence with at least 90%, at least 91%, at least 92%, at least 93%, at least 94%, at least 95%, at least 96%, at least 97%, at least 98%, or at least 99% sequence identity thereof; and wherein one primary nucleic acid sequence part encodes SEQ ID NO: 4 (SARS-COV-2 M) or an amino acid sequence with at least 90%, at least 91%, at least 92%, at least 93%, at least 94%, at least 95%, at least 96%, at least 97%, at least 98%, or at least 99% sequence identity thereof.

In some embodiments, the invention relates to a nucleic acid sequence described herein, wherein a) comprises two primary nucleic acid sequence parts, wherein one primary nucleic acid sequence part encodes SEQ ID NO: 2 (SARS-COV-2 S) or an amino acid sequence with at least 90%, at least 91%, at least 92%, at least 93%, at least 94%, at least 95%, at least 96%, at least 97%, at least 98%, or at least 99% sequence identity thereof; and wherein one primary nucleic acid sequence part encodes SEQ ID NO: 3 (SARS-COV-2 E) or an amino acid sequence with at least 90%, at least 91%, at least 92%, at least 93%, at least 94%, at least 95%, at least 96%, at least 97%, at least 98%, or at least 99% sequence identity thereof.

In some embodiments, the invention relates to a nucleic acid sequence described herein, wherein a) comprises two primary nucleic acid sequence parts, wherein one primary nucleic acid sequence part encodes SEQ ID NO: 2 (SARS-COV-2 S) or an amino acid sequence with at least 90%, at least 91%, at least 92%, at least 93%, at least 94%, at least 95%, at least 96%, at least 97%, at least 98%, or at least 99% sequence identity thereof; and wherein one primary nucleic acid sequence part encodes SEQ ID NO: 4 (SARS-COV-2 M) or an amino acid sequence with at least 90%, at least 91%, at least 92%, at least 93%, at least 94%, at least 95%, at least 96%, at least 97%, at least 98%, or at least 99% sequence identity thereof.

In some embodiments, the invention relates to a nucleic acid sequence described herein, wherein a) comprises two primary nucleic acid sequence parts, wherein one primary nucleic acid sequence part encodes SEQ ID NO: 3 (SARS-COV-2 E) or an amino acid sequence with at least 90%, at least 91%, at least 92%, at least 93%, at least 94%, at least 95%, at least 96%, at least 97%, at least 98%, or at least 99% sequence identity thereof; and wherein one primary nucleic acid sequence part encodes SEQ ID NO: 4 (SARS-COV-2 M) or an amino acid sequence with at least 90%, at least 91%, at least 92%, at least 93%, at least 94%, at least 95%, at least 96%, at least 97%, at least 98%, or at least 99% sequence identity thereof.

The nucleic acid according to the invention allows to significantly accelerate the production of a virus, virus part and/or virus particle that can be used for example in research or in a pharmaceutical composition such as a vaccine. These the mentioned vaccines and leads to well-defined vaccines which are very specific to a virus or a variant thereof, especially to the coronavirus SARS-COV-2.

The resulting sequence-defined genome is produced by PCR-driven molecular biology techniques, which allow to introduce deletions of genes and regulatory elements, which the virus needs for its genetic reproduction and replication.

The intended deletions can be designed in a way that they do not retain any sequence-overlap with those plasmids, in which the respective viral genes are expressed inside the producer cell line.

The nucleic acid according to the invention differs from fully-synthetic sequences produced by chemical synthesis and allows the biologic production of the nucleic acid by means of molecular biology techniques.

The fact that the protein components can be produced using common expression systems used for protein expression means that vaccines can be made available in large quantities very quickly. This is of crucial importance for viruses such as the coronavirus SARS-COV-2, whose spread has assumed the proportions of a pandemic and whose containment, therefore, requires widespread vaccine administration.

The invention provides a combinatoric approach wherein certain deletions, omissions or dysfunctions enable the efficient production of replication-limited virus particles with high antigenicity. In some embodiments the primary nucleic acid sequence part that encodes for SARS-COV-2 E is deleted, dysfunction or not present in the nucleic acid sequence of the invention. Therefore, the inventors found that certain sequence parts of the SARS-COV-2 genome (or sequences having equivalent functions) can be combined or omitted for the efficient production of replication limited virus particles.

The inventors found that two or three primary nucleic sequence part as described herein are required for high antigenicity. These can be combined in any combination with the secondary nucleic acid sequence parts described herein. These combinations may comprise single or double deletions/dysfunction/omission of the secondary nucleic acid sequences.

Missing sequence parts may limit reproducibility and may be complemented in a production system to enable efficient production.

The inventors found, that the nucleic acid of the invention wherein the function of a SARS-COV-2 amino acid sequence encoded by ORF3a is deleted or not present and which does not include a primary nucleic acid sequence part that encodes for SARS-CoV-2 E, is particularly useful, if further sequence parts are not present, deleted or dysfunctional. Therefore, a triple (or more) deletion of encoding elements compared to the original SARS-COV-2 virus genome is more useful than a double deletion of ORF3a and E. In some embodiments, the nucleic acid of the invention comprises an ORF3a/E double deletion and a further deletion of the function selected from the group of ORF6 deletion, ORF8 deletion, ORF7a deletion, M deletion, S deletion and N deletion, when compared to the sequences encoded by SARS-COV-2.

The reproducibility of the nucleic acid sequence is reduced by omitting functional sequence parts that play a crucial role in virus reproduction. These parts can be omitted e.g. by not being synthesized, by being deleted or by being made dysfunctional. As such the SARS-COV-2 can be efficiently produced in specialized cells but have no or a limited ability to reproduce in other cells.

Immunity derived from infection with SARS-COV-2 has proven to provide more protection than immunity derived from SARS-COV-2 S protein mediated vaccination (see e.g., Gazit, Sivan, et al., 2021, medRxiv). The sequences encode a combination of structural proteins of the wild-type virus or proteins with equivalent functions thereof. This enables a broad range of epitopes available to the immune system including T-cell epitopes (see, e.g., Grifoni, A., et al., 2020, Cell, 181(7), 1489-1501). This broad range of epitopes may enable immunity against a broad range of virus variants in patients with or without pre-existing immunity.

Accordingly, the invention is at least in part based on the discovery that the nucleic acid of the invention, enables efficient production of a combination virus-like proteins with limited replication capabilities but similar antigenic effect to the original virus.

In certain embodiments, the invention relates to the nucleic acid sequence of the invention, wherein the nucleic acid sequence comprises two or three primary nucleic acid sequence parts, wherein a primary nucleic acid sequence part encodes an amino acid sequence selected from the group consisting of i) SEQ ID NO: 1 (SARS-COV-2 N) or an amino acid sequence with at least 90% sequence identity thereof; ii) SEQ ID NO: 2 (SARS-COV-2 S) or an amino acid sequence with at least 90% sequence identity thereof; and iii) SEQ ID NO: 3 (SARS-COV-2 E) or an amino acid sequence with at least 90% sequence identity thereof; and wherein the nucleic acid sequence has no sequence part that encodes an amino acid sequence having the function of a SARS-CoV-2 amino acid sequence encoded by SEQ ID NO: 4 (SARS-COV-2 M).

In certain embodiments, the invention relates to the nucleic acid sequence of the invention, wherein the nucleic acid sequence comprises three primary nucleic acid sequence parts: i) SEQ ID NO: 1 (SARS-COV-2 N) or an amino acid sequence with at least 90% sequence identity thereof; ii) SEQ ID NO: 2 (SARS-COV-2 S) or an amino acid sequence with at least 90% sequence identity thereof; and iii) SEQ ID NO: 3 (SARS-COV-2 E) or an amino acid sequence with at least 90% sequence identity thereof.

Therefore, nucleic acid sequence of the invention in this embodiment can comprise any other sequence part(s) or all other parts of the SARS-COV-2 genome but no sequence part encoding an amino acid sequence having the function of a SARS-COV-2 amino acid sequence encoded by SEQ ID NO: 4 (SARS-COV-2 M). For example, sequence having the function of a SARS-COV-2 amino acid sequence encoded by SEQ ID NO: 4 (SARS-COV-2 M) is not present, deleted, or dysfunctional.

In certain embodiments, the invention relates to the nucleic acid sequence of the invention, wherein the nucleic acid sequence comprises no nucleic acid sequence part encodes an amino acid sequence having the function of a SARS-COV-2 amino acid sequence encoded by ORF7 and ORF8.

Therefore, nucleic acid sequence of the invention in this embodiment can comprise any other sequence part(s) or all other parts of the SARS-COV-2 genome but no sequence part encoding an amino acid sequence having the function of a SARS-COV-2 amino acid sequence encoded by SEQ ID NO: 4 (SARS-COV-2 M), ORF7 and/or ORF8. For example, sequence having the function of a SARS-COV-2 amino acid sequence encoded by SEQ ID NO: 4 (SARS-COV-2 M), ORF7 and ORF8 are not present, deleted, dysfunctional or a combination thereof (e.g. SEQ ID NO: 4 (SARS-CoV-2 M) not present and ORF7 and ORF8 dysfunctional).

In certain embodiments, the invention relates to the nucleic acid sequence of the invention, wherein the nucleic acid sequence comprises no nucleic acid sequence part encodes an amino acid sequence having the function of a SARS-COV-2 amino acid sequence encoded by ORF6 and ORF7ab.

Therefore, nucleic acid sequence of the invention in this embodiment can comprise any other sequence part(s) or all other parts of the SARS-COV-2 genome but no sequence part encoding an amino acid sequence having the function of a SARS-COV-2 amino acid sequence encoded by SEQ ID NO: 4 (SARS-COV-2 M), ORF6 and/or ORF7ab. For example, sequence having the function of a SARS-COV-2 amino acid sequence encoded by SEQ ID NO: 4 (SARS-COV-2 M), ORF6 and ORF7ab are not present, deleted, dysfunctional or a combination thereof (e.g. SEQ ID NO: 4 (SARS-CoV-2 M) not present and ORF6 and ORF7ab dysfunctional).

In certain embodiments, the invention relates to the nucleic acid sequence of the invention, wherein the nucleic acid sequence comprises no nucleic acid sequence part encodes an amino acid sequence having the function of a SARS-COV-2 amino acid sequence encoded by ORF6, ORF7ab and ORF8.

Therefore, nucleic acid sequence of the invention in this embodiment can comprise any other sequence part(s) or all other parts of the SARS-COV-2 genome but no sequence part encoding an amino acid sequence having the function of a SARS-COV-2 amino acid sequence encoded by SEQ ID NO: 4 (SARS-COV-2 M), ORF6, ORF7ab and/or ORF8. For example, a sequence having the function of a SARS-COV-2 amino acid sequence encoded by SEQ ID NO: 4 (SARS-COV-2 M), ORF6, ORF7ab and ORF8 are not present, deleted, dysfunctional or a combination thereof (e.g. SEQ ID NO: 4 (SARS-COV-2 M) not present and ORF6, ORF7ab and ORF8 dysfunctional).

In certain embodiments, the invention relates to the nucleic acid sequence of the invention, wherein the nucleic acid sequence comprises a primary nucleic acid sequence part encoding an amino acid sequence as defined by SEQ ID NO: 1 (SARS-CoV-2 N) (or an amino acid sequence with at least 90% sequence identity thereof), a secondary nucleic acid sequence part encoding an amino acid sequence having the function of a SARS-COV-2 amino acid sequence encoded by ORF3a and a sequence part of the nucleic acid sequence located between said primary nucleic acid sequence part and said secondary nucleic acid sequence part, wherein the sequence part comprises I) SEQ ID NO: 35 or a sequence having at least 90% sequence identity to SEQ ID NO: 35.

In certain embodiments, the invention relates to the nucleic acid sequence of the invention, wherein the nucleic acid sequence comprises a primary nucleic acid sequence part encoding an amino acid sequence as defined by SEQ ID NO: 1 (SARS-CoV-2 N) (or an amino acid sequence with at least 90% sequence identity thereof), a secondary nucleic acid sequence part encoding an amino acid sequence having the function of a SARS-COV-2 amino acid sequence encoded by ORF3a and a sequence part of the nucleic acid sequence located between said primary nucleic acid sequence part and said secondary nucleic acid sequence part, wherein the sequence part comprises II) SEQ ID NO: 36 or a sequence having at least 90% sequence identity to SEQ ID NO: 36.

In certain embodiments, the invention relates to the nucleic acid sequence of the invention, wherein the nucleic acid sequence comprises a primary nucleic acid sequence part encoding an amino acid sequence as defined by SEQ ID NO: 1 (SARS-CoV-2 N) (or an amino acid sequence with at least 90% sequence identity thereof), a secondary nucleic acid sequence part encoding an amino acid sequence having the function of a SARS-COV-2 amino acid sequence encoded by ORF3a and a sequence part the nucleic acid sequence located between said primary nucleic acid sequence part and said secondary nucleic acid sequence part, wherein the sequence part comprises III) SEQ ID NO:37 or a sequence having at least 90% sequence identity to SEQ ID NO: 37.

In certain embodiments, the invention relates to a biologically produced nucleic acid sequence comprising two or three nucleic acid sequence parts encoding an amino acid sequence selected from the group consisting of: i) SEQ ID NO: 1 (SARS-COV-2 N) or an amino acid sequence with at least 90% sequence identity thereof; ii) SEQ ID NO: 2 (SARS-COV-2 S) or an amino acid sequence with at least 90% sequence identity thereof; and iii) SEQ ID NO: 3 (SARS-COV-2 E) or an amino acid sequence with at least 90% sequence identity thereof; and wherein the nucleic acid sequence has no sequence part that encodes an amino acid sequence having the function of a SARS-CoV-2 amino acid sequence encoded by SEQ ID NO: 4 (SARS-COV-2 M).

In certain embodiments, the invention relates to the nucleic acid sequence of the invention, wherein the nucleic acid sequence comprises a sequence as defined by SEQ ID NO: 33. For example a sequence as defined by SEQ ID NO: 33 that is located between a nucleic acid sequence part encoding an amino acid sequence as defined by SEQ ID NO: 1 (SARS-COV-2 N) (or an amino acid sequence with at least 90% sequence identity thereof) and a nucleic acid sequence part encoding an amino acid sequence having the function of a SARS-COV-2 amino acid sequence encoded by ORF3a.

In certain embodiments, the invention relates to the nucleic acid sequence of the invention, wherein the nucleic acid sequence comprises two primary nucleic acid sequence parts: i) SEQ ID NO: 1 (SARS-COV-2 N) or an amino acid sequence with at least 90% sequence identity thereof; and ii) SEQ ID NO: 2 (SARS-COV-2 S) or an amino acid sequence with at least 90% sequence identity thereof; and wherein the nucleic acid sequence has no sequence part that encodes an amino acid sequence having the function of a SARS-COV-2 amino acid sequence encoded by SEQ ID NO: 4 (SARS-COV-2 M) and SEQ ID NO: 3 (SARS-COV-2 E).

In certain embodiments, the invention relates to the nucleic acid sequence of the invention, wherein the nucleic acid sequence comprises a sequence as defined by SEQ ID NO: 34. Such a sequence is, for example, a sequence as defined by SEQ ID NO: 34 that is located between a nucleic acid sequence part encoding an amino acid sequence as defined by SEQ ID NO: 1 (SARS-COV-2 N) (or an amino acid sequence with at least 90% sequence identity thereof) and a nucleic acid sequence part encoding an amino acid sequence having the function of a SARS-COV-2 amino acid sequence encoded by ORF3a. In certain embodiments, the invention relates to the nucleic acid sequence of the invention, wherein the nucleic acid sequence comprises a sequence as defined by SEQ ID NO: 35. Such a sequence is, for example, a sequence as defined by SEQ ID NO: 35 that is located between a nucleic acid sequence part encoding an amino acid sequence as defined by SEQ ID NO: 1 (SARS-COV-2 N) (or an amino acid sequence with at least 90% sequence identity thereof) and a nucleic acid sequence part encoding an amino acid sequence having the function of a SARS-COV-2 amino acid sequence encoded by ORF3a.

In certain embodiments, the invention relates to the nucleic acid sequence of the invention, wherein the nucleic acid sequence comprises a sequence as defined by SEQ ID NO: 36. Such a sequence is, for example, a sequence as defined by SEQ ID NO: 36 that is located between a nucleic acid sequence part encoding an amino acid sequence as defined by SEQ ID NO: 1 (SARS-COV-2 N) (or an amino acid sequence with at least 90% sequence identity thereof) and a nucleic acid sequence part encoding an amino acid sequence having the function of a SARS-COV-2 amino acid sequence encoded by ORF3a.

In certain embodiments, the invention relates to the nucleic acid sequence of the invention, wherein the nucleic acid sequence comprises a sequence as defined by SEQ ID NO: 37. Such a sequence is, for example, a sequence as defined by SEQ ID NO: 37 that is located between a nucleic acid sequence part encoding an amino acid sequence as defined by SEQ ID NO: 1 (SARS-COV-2 N) (or an amino acid sequence with at least 90% sequence identity thereof) and a nucleic acid sequence part encoding an amino acid sequence having the function of a SARS-COV-2 amino acid sequence encoded by ORF3a.

In certain embodiments, the invention relates to the nucleic acid sequence of the invention, wherein the nucleic acid sequence comprises a sequence as defined by SEQ ID NO: 66. Such a sequence is, for example, a sequence as defined by SEQ ID NO: 66 that is located between a nucleic acid sequence part encoding an amino acid sequence as defined by SEQ ID NO: 1 (SARS-COV-2 N) (or an amino acid sequence with at least 90% sequence identity thereof) and a nucleic acid sequence part encoding an amino acid sequence having the function of a SARS-COV-2 amino acid sequence encoded by ORF3a.

In certain embodiments, the invention relates to the nucleic acid sequence of the invention, wherein the nucleic acid sequence comprises a sequence as defined by SEQ ID NO: 67. Such a sequence is, for example, a sequence as defined by SEQ ID NO: 67 that is located between a nucleic acid sequence part encoding an amino acid sequence as defined by SEQ ID NO: 1 (SARS-COV-2 N) (or an amino acid sequence with at least 90% sequence identity thereof) and a nucleic acid sequence part encoding an amino acid sequence having the function of a SARS-COV-2 amino acid sequence encoded by ORF3a.

The person skilled in the art is aware that the sequence parts as defined by SEQ ID NO: 34-37 are typically located between the sequence parts described herein, wherein further parts of the sequence correspond to a SARS-COV-2 genome or a SARS-COV-2 variant thereof. As such the SEQ ID NO: 34-37 provide a way to introduce deletions in the SARS-COV-2 genome. Further deletions, insertions and/or replacements may be introduced in the adjacent sequence parts and/or in the further parts of the sequence.

The inventors demonstrated that, after multiple cell passages of AM, AEAM, AMAORF7AORF8, AMAORF6AORF7ab and/or AMAORF6AORF7AORF8 viruses (see Example 9) in a genetically engineered producer cell authentic sequence can be retained. Furthermore, the inventors showed that in several parallel infections of the vaccine viruses and after multiple blind-passages on VeroE6 cells, no viable virus emerges and already after the first passage no replicative virus can be demonstrated in normal, SARS-COV-2 susceptible cells. The inventors furthermore demonstrated that sequential passages in permissive producer cells, the population of offspring vaccine virus is found to be well conserved.

Accordingly, the invention is at least in part based on the finding that the nucleic acid described herein can encode a virus with a low chance of spontaneous change during virus propagation in cell culture in the producer cells, while being unlikely to re-generate infectious, replication-competent wild-type or wild-type-like SARS-COV-2. As such the nucleic acid described herein enables safe production of a SARS-COV-2-like antigens and/or vaccines.

In certain embodiments, the invention relates to the nucleic acid sequence of the invention, wherein for the secondary nucleic acid sequence part encoding an amino acid sequence having the function of a SARS-COV-2 amino acid sequence ORF3a is a sequence defined by SEQ ID NO: 5 or a sequence having with at least 90%, at least 91%, at least 92%, at least 93%, at least 94%, at least 95%, at least 96%, at least 97%, at least 98%, or at least 99% sequence identity thereof, that retains the function of a SARS-COV-2 amino acid sequence encoded by ORF3a.

In certain embodiments, the invention relates to the nucleic acid sequence of the invention, wherein for the secondary nucleic acid sequence part encoding an amino acid sequence having the function of a SARS-COV-2 amino acid sequence ORF6 is a sequence defined by SEQ ID NO: 6 or a sequence having with at least 90%, at least 91%, at least 92%, at least 93%, at least 94%, at least 95%, at least 96%, at least 97%, at least 98%, or at least 99% sequence identity thereof, that retains the function of a SARS-COV-2 amino acid sequence encoded by ORF6.

In certain embodiments, the invention relates to the nucleic acid sequence of the invention, wherein for the secondary nucleic acid sequence part encoding an amino acid sequence having the function of a SARS-COV-2 amino acid sequence ORF7a is a sequence defined by SEQ ID NO: 7 or a sequence having with at least 90%, at least 91%, at least 92%, at least 93%, at least 94%, at least 95%, at least 96%, at least 97%, at least 98%, or at least 99% sequence identity thereof, that retains the function of a SARS-COV-2 amino acid sequence encoded by ORF7a.

In certain embodiments, the invention relates to the nucleic acid sequence of the invention, wherein for the secondary nucleic acid sequence part encoding an amino acid sequence having the function of a SARS-COV-2 amino acid sequence ORF7b is a sequence defined by SEQ ID NO: 8 or a sequence having with at least 90%, at least 91%, at least 92%, at least 93%, at least 94%, at least 95%, at least 96%, at least 97%, at least 98%, or at least 99% sequence identity thereof, that retains the function of a SARS-COV-2 amino acid sequence encoded by ORF7b.

In certain embodiments, the invention relates to the nucleic acid sequence of the invention, wherein for the secondary nucleic acid sequence part encoding an amino acid sequence having the function of a SARS-COV-2 amino acid sequence ORF8 is a sequence defined by SEQ ID NO: 9 or a sequence having with at least 90%, at least 91%, at least 92%, at least 93%, at least 94%, at least 95%, at least 96%, at least 97%, at least 98%, or at least 99% sequence identity thereof, that retains the function of a SARS-COV-2 amino acid sequence encoded by ORF8.

In certain embodiments, the invention relates to the nucleic acid sequence of the invention, wherein for the secondary nucleic acid sequence part encoding an amino acid sequence having the function of a SARS-COV-2 amino acid sequence i) ORF3a is a sequence defined by SEQ ID NO: 5; ii) ORF6 is a sequence defined by SEQ ID NO: 6; iii) ORF7a is a sequence defined by SEQ ID NO: 7; and/or iv) ORF8 is a sequence defined by SEQ ID NO: 9.

The inventors found, that ORFs largely identical to the sequences of the SARS-COV-2 enable efficient production of SARS-COV-2 proteins.

Accordingly, the invention is at least in part based on the finding that SARS-COV-2 proteins can be produced efficiently, as described herein.

In certain embodiments, the invention relates to the nucleic acid sequence of the invention, wherein the nucleic acid sequence comprises three primary nucleic acid sequence parts.

In some embodiments, the invention relates to a nucleic acid sequence described herein, wherein a) comprises three primary nucleic acid sequence parts, wherein one primary nucleic acid sequence part encodes SEQ ID NO: 1 (SARS-COV-2 N) or an amino acid sequence with at least 90%, at least 91%, at least 92%, at least 93%, at least 94%, at least 95%, at least 96%, at least 97%, at least 98%, or at least 99% sequence identity thereof; wherein one primary nucleic acid sequence part encodes SEQ ID NO: 2 (SARS-COV-2 S) or an amino acid sequence with at least 90%, at least 91%, at least 92%, at least 93%, at least 94%, at least 95%, at least 96%, at least 97%, at least 98%, or at least 99% sequence identity thereof; and wherein one primary nucleic acid sequence part encodes SEQ ID NO: 3 (SARS-COV-2 E) or an amino acid sequence with at least 90%, at least 91%, at least 92%, at least 93%, at least 94%, at least 95%, at least 96%, at least 97%, at least 98%, or at least 99% sequence identity thereof.

In some embodiments, the invention relates to a nucleic acid sequence described herein, wherein a) comprises three primary nucleic acid sequence parts, wherein one primary nucleic acid sequence part encodes SEQ ID NO: 1 (SARS-COV-2 N) or an amino acid sequence with at least 90%, at least 91%, at least 92%, at least 93%, at least 94%, at least 95%, at least 96%, at least 97%, at least 98%, or at least 99% sequence identity thereof; wherein one primary nucleic acid sequence part encodes SEQ ID NO: 2 (SARS-COV-2 S) or an amino acid sequence with at least 90%, at least 91%, at least 92%, at least 93%, at least 94%, at least 95%, at least 96%, at least 97%, at least 98%, or at least 99% sequence identity thereof; and wherein one primary nucleic acid sequence part encodes SEQ ID NO: 4 (SARS-COV-2 M) or an amino acid sequence with at least 90%, at least 91%, at least 92%, at least 93%, at least 94%, at least 95%, at least 96%, at least 97%, at least 98%, or at least 99% sequence identity thereof.

In some embodiments, the invention relates to a nucleic acid sequence described herein, wherein a) comprises three primary nucleic acid sequence parts, wherein one primary nucleic acid sequence part encodes SEQ ID NO: 1 (SARS-COV-2 N) or an amino acid sequence with at least 90%, at least 91%, at least 92%, at least 93%, at least 94%, at least 95%, at least 96%, at least 97%, at least 98%, or at least 99% sequence identity thereof; wherein one primary nucleic acid sequence part encodes SEQ ID NO: 3 (SARS-COV-2 E) or an amino acid sequence with at least 90%, at least 91%, at least 92%, at least 93%, at least 94%, at least 95%, at least 96%, at least 97%, at least 98%, or at least 99% sequence identity thereof; and wherein one primary nucleic acid sequence part encodes SEQ ID NO: 4 (SARS-COV-2 M) or an amino acid sequence with at least 90%, at least 91%, at least 92%, at least 93%, at least 94%, at least 95%, at least 96%, at least 97%, at least 98%, or at least 99% sequence identity thereof.

In some embodiments, the invention relates to a nucleic acid sequence described herein, wherein a) comprises three primary nucleic acid sequence parts, wherein one primary nucleic acid sequence part encodes SEQ ID NO: 2 (SARS-COV-2 S) or an amino acid sequence with at least 90%, at least 91%, at least 92%, at least 93%, at least 94%, at least 95%, at least 96%, at least 97%, at least 98%, or at least 99% sequence identity thereof; wherein one primary nucleic acid sequence part encodes SEQ ID NO: 3 (SARS-COV-2 E) or an amino acid sequence with at least 90%, at least 91%, at least 92%, at least 93%, at least 94%, at least 95%, at least 96%, at least 97%, at least 98%, or at least 99% sequence identity thereof; and wherein one primary nucleic acid sequence part encodes SEQ ID NO: 4 (SARS-COV-2 M) or an amino acid sequence with at least 90%, at least 91%, at least 92%, at least 93%, at least 94%, at least 95%, at least 96%, at least 97%, at least 98%, or at least 99% sequence identity thereof.

Therefore, in some embodiments, the sequences encode a combination of three structural proteins of the wild-type virus or proteins with equivalent functions thereof. This enables a broad range of epitopes available to the immune system including T-cell epitopes (see, e.g., Grifoni, A., et al., 2020, Cell, 181 (7), 1489-1501). This broad range of epitopes may enable immunity against a broad range of virus variants in patients with or without pre-existing immunity.

Accordingly, the invention is at least in part based on the discovery that the nucleic acid of the invention, enables efficient production of a combination virus-like proteins with limited replication capabilities but similar antigenic effect to the original virus.

In certain embodiments, the invention relates to the nucleic acid sequence of any one of the invention, wherein one of the secondary nucleic acid sequence parts encodes an amino acid sequence having the function of a SARS-COV-2 amino acid sequence encoded by ORF3a.

In certain embodiments, the invention relates to the nucleic acid sequence of any one of the invention, wherein one of the secondary nucleic acid sequence parts encodes an amino acid sequence having the function of a SARS-COV-2 amino acid sequence encoded by ORF3a and one of the secondary nucleic acid sequence parts encodes an amino acid sequence having the function of a SARS-COV-2 amino acid sequence encoded by ORF7b.

The inventors found, that ORF3a facilitates production of SARS-COV-2 proteins that can be used in a vaccine.

Accordingly, the invention is at least in part based on the finding that ORF3a contributes to the production of SARS-COV-2 proteins as described herein.

In certain embodiments, the invention relates to the nucleic acid sequence of any one of the invention, wherein the primary nucleic acid sequence parts and the secondary nucleic acid sequence parts are ordered in 5′ to 3′ direction in the following order:
1. SEQ ID NO: 2 (SARS-COV-2 S) or an amino acid sequence with at least 90%, at least 91%, at least 92%, at least 93%, at least 94%, at least 95%, at least 96%, at least 97%, at least 98%, or at least 99% sequence identity thereof,
2. nucleic acid sequence part encoding an amino acid sequence having the function of a SARS-COV-2 amino acid sequence encoded by ORF3a;
3. SEQ ID NO: 3 (SARS-COV-2 E) or an amino acid sequence with at least 90%, at least 91%, at least 92%, at least 93%, at least 94%, at least 95%, at least 96%, at least 97%, at least 98%, or at least 99% sequence identity thereof,
4. SEQ ID NO: 4 (SARS-COV-2 M) or an amino acid sequence with at least 90%, at least 91%, at least 92%, at least 93%, at least 94%, at least 95%, at least 96%, at least 97%, at least 98%, or at least 99% sequence identity thereof,
5. nucleic acid sequence part encoding an amino acid sequence having the function of a SARS-COV-2 amino acid sequence encoded by ORF6,
6. nucleic acid sequence part encoding an amino acid sequence having the function of a SARS-COV-2 amino acid sequence encoded by ORF7a,
7. nucleic acid sequence part encoding an amino acid sequence encoding an amino acid sequence having the function of a SARS-COV-2 amino acid sequence encoded by ORF8,
8. SEQ ID NO: 1 (SARS-COV-2 N) or an amino acid sequence with at least 90%, at least 91%, at least 92%, at least 93%, at least 94%, at least 95%, at least 96%, at least 97%, at least 98%, or at least 99% sequence identity thereof.

The inventors found that the sequences described herein can be expressed particularly effective, if the order of the sequence parts corresponds to the original order of the SARS-COV-2 genome sequence. As such, the order can shift to the next corresponding sequence, if one or more of the sequence parts 1. -9. is not present, deleted, or dysfunctional. Furthermore, the sequence parts do not need to be directly linked, but may also have other sequences in between or overlapping sequence parts. The order described herein may be considered as having a later starting point than in 5′ to 3′ direction, if the order number is higher.

Accordingly, the invention is at least in part based on the finding that the nucleic acid can be particularly efficiently expressed if the sequence parts are ordered as described herein.

In some embodiments, the nucleic acid sequence of the invention comprises further sequence parts e.g. SARS-COV-2 sequence parts such as ORF1a, ORF1b, ORF1ab and/or ORF10.

In certain embodiments, the invention relates to the nucleic acid sequence of any one of the invention, wherein the nucleic acid sequence comprises a nucleic acid sequence defined by the SEQ ID NO: 10 (SARS-COV-2 genome) or a sequence with at least 90%, at least 91%, at least 92%, at least 93%, at least 94%, at least 95%, at least 96%, at least 97%, at least 98%, or at least 99% sequence identity thereof with a deletion and/or a dysfunctionality of the E gene, ORF6 gene and ORF8 gene.

In certain embodiments, the invention relates to the nucleic acid sequence of any one of the invention, wherein the nucleic acid sequence comprises a nucleic acid sequence defined by the SEQ ID NO: 10 (SARS-COV-2 genome) or a sequence with at least 90%, at least 91%, at least 92%, at least 93%, at least 94%, at least 95%, at least 96%, at least 97%, at least 98%, or at least 99% sequence identity thereof with a deletion and/or a dysfunctionality of the E gene, ORF6 gene, ORF7a gene and ORF8 gene.

The term “E gene”, as used herein, refers to a nucleic sequence encoding for SEQ ID NO: 15.

The term “ORF6 gene”, as used herein, refers to a nucleic sequence encoding for SEQ ID NO: 6.

The term “ORF7a gene”, as used herein, refers to a nucleic sequence encoding for SEQ ID NO: 7.

The term “ORF8 gene”, as used herein, refers to a nucleic sequence encoding for SEQ ID NO: 9.

The inventors found that the E-gene, ORF6 gene, ORF7a and/or ORF8 gene can be removed and at least partially replaced by trans-complementary producer cells. This enables efficient and safe production of replication-limited virus particles.

Accordingly, the invention is at least in part based on the finding that removing the functionality of the E-gene, ORF6 gene, ORF7a and/or ORF8 gene is particularly useful in the production of replication-limited virus particles.

In certain embodiments, the invention relates to a vector comprising the nucleic acid sequence of one of the invention.

The term “vector”, as used herein, refers to a nucleic acid molecule, capable of transferring or transporting itself and/or another nucleic acid molecule into a cell. The transferred nucleic acid is generally linked to, i.e., inserted into, the vector nucleic acid molecule. A vector may include sequences that direct autonomous replication in a cell, or may include sequences sufficient to allow integration into host cell DNA. In some embodiments, the vector described herein is a vector selected from the group of plasmids (e.g., DNA plasmids or RNA plasmids), shuttle vectors, transposons, cosmids, artificial chromosomes (e.g. bacterial, yeast, human), and viral vectors. In some embodiments, the invention relates to a vector according to the invention, wherein the vector comprises at least one sequence encoding an T7 promoter and at least two untranslated regions that contain sequences that enable the synthesis of negative-strand RNA and/or that enable positive-strand RNA synthesis.

In certain embodiments, the invention relates to the vector of the invention, wherein the vector is a plasmid vector.

In some embodiments, the plasmid vector described herein has a selection marker and sequence determining the origin of replication.

The inventors found that plasmid vectors are particularly suitable for transferring large sequences as described herein.

Accordingly, the invention is at least in part based on the finding that plasmid vectors are particularly effective for transferring the nucleic acid sequence as described herein.

In certain embodiments, the invention relates to the vector of the invention, wherein the vector comprises a sequence as defined by SEQ ID NO: 11 (biologically produced vector with ORF7a gene) or a sequence having 90%, at least 91%, at least 92%, at least 93%, at least 94%, at least 95%, at least 96%, at least 97%, at least 98%, or at least 99% sequence identity thereof.

In certain embodiments, the invention relates to the vector of the invention, wherein the vector comprises a sequence as defined by SEQ ID NO: 12 (biologically produced vector without ORF7a gene) or a sequence having 90%, at least 91%, at least 92%, at least 93%, at least 94%, at least 95%, at least 96%, at least 97%, at least 98%, or at least 99% sequence identity thereof.

In some embodiments, the vector described herein is used in combination with at least one transfection enhancer, e.g., a transfection enhancer selected from the group of oligonucleotides, lipoplexes, polymersomes, polyplexes, dendrimers, inorganic nanoparticles and cell-penetrating peptides.

The vector described herein can be used for efficient transfer and/or amplification of the nucleic acid sequence of the invention in an amplifying host cell.

The product of the amplification in an amplifying host cell (e.g., yeast cells) may be isolated and subsequently translated in a further host cell (e.g., human cell).

Accordingly, the invention is at least in part based on the discovery that the vector described herein enables efficient amplification of the nucleic acids described herein and efficient production of a combination virus-like proteins with limited replication capabilities but high antigenicity. The inventive nucleic acids lead through the above procedure to the production of a dispersion comprising proteins and other building blocks.

Suitable separation methods known to the person skilled in the art, such as centrifugation or chromatography, can be used to separate these building blocks, if necessary also from residues of the production cell line used or other production aids or organisms, and thus purify them.

In some embodiments, the building blocks described herein are purified using at least one separation method selected from the group of chromatography, precipitation, ultracentrifugation, tangential-flow filtration, and enzymatic digestion

These optionally purified virus envelopes or fragments thereof represent the basis of the vaccine, which is then transferred into different dosage forms depending on the type of application.

Typically, an adjuvant is used for this purpose, stabilizers to improve shelf-life, salts and buffers. The vaccines are thus the product of the long-chain, fully synthetic nucleic acids described here.

In certain embodiments, the invention relates to a host cell comprising the nucleic acid sequence of the invention or the vector of the invention.

The term “host cell”, as used herein, refers to a cell into which exogenous nucleic acid has been introduced, including the progeny of such a cell. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

In some embodiments, the host cell described herein comprises a cell that allows viral entry of SARS-COV-2. In some embodiments, the host cell described herein comprises a cell that expresses the human ACE2 receptor or a functional human-like ACE2 receptor. The human-like ACE2 receptors that allow viral entry of SARS-COV-2 are known to the person skilled in the art (see, e.g., Damas, J., et al., 2020, Proceedings of the National Academy of Sciences, 117 (36), 22311-22322).

In some the host cell described herein comprises at least one cell type selected from the group of HEK293, MDCK, Chinese hamster ovary (CHO), SF9, Vero, MRC 5, Per.C6, PMK, and WI-38.

In some embodiments, the host cell described herein comprises a cell that is at least partially human or a cell of an at least partially human cell line.

In some embodiments, the host cell described herein comprises a cell that allows the production of a viral particle comprising the nucleotide of the invention or the vector of the invention that is selectively replicable in it is fully replicable in cells of the host cell but not or unsubstantially in cells of the human body. This selective replicability is achieved by cells that comprise complementary proteins for the replication of the viral particle.

In some embodiments, the host cell described herein comprises a cell that can express at least one protein for viral replication. In some embodiments, the host cell described herein comprises a cell that can express at least one protein component for viral replication that is not encoded in the nucleotide acid sequence of the invention or the vector of the invention.

Transduction of host cells by the vector of the invention can be achieved by stable or transient transduction (see, e.g., Stepanenko, A. A., and Heng, H. H., 2017, Mutation Research/Reviews in Mutation Research, 773, 91-103).

If DNA is introduced into the production unit according to a first embodiment, this is usually done using a plasmid suitable for this purpose.

Alternatively, the DNA may be introduced into the host cell by any kind of vector.

In certain embodiments, the invention relates to the host cell of the invention additionally comprising at least one complementary SARS-COV-2 sequence thereof.

The term “complementary SARS-COV-2 sequence”, as used herein, refers to a sequence having the function of a SARS-COV-2 protein, wherein the function is not comprised in the nucleic acid it complements. Therefore, the term “complementary” as used herein is not referring to the ability to form a double stranded structure, but rather refers to a nucleic acid sequence that encodes an additional a SARS-COV-2 protein or a protein with the function of an additional SARS-COV-2 protein.-For example, a sequence is complementary to the sequence as defined by SEQ ID NO: 12, if it comprises a nucleotide acid sequence encoding a functional SARS-COV-2 E, ORF6, ORF7a and/or ORF8 protein.

In some embodiments, the nucleic acid of the invention combined with all complementary sequences comprised in the host cell contain herein completes at least 2, at least 3, at least 4, at least 5, at least 6, at least 7, at least 8 at least 9 or all sequence parts selected from the group of ORF1a, ORF1b, S, ORF3a, E, M, ORF6, ORF7a, ORF8, N.

In certain embodiments, the invention relates to the host cell of the invention additionally comprising at least one nucleic acid sequence that a) is not comprised in the vector of the invention or the nucleic acid of the invention; and b) comprises at least one sequence part that encodes a function of a protein encoded in the SARS-COV-2 genome.

The sequence additionally comprised in the host cell may be added to the host cell by a separate vector such as a plasmid vector.

The inventors found that the nucleic sequences described herein can be used as part of a trans-complementary production system.

Accordingly, the invention is at least in part based on the finding that trans-complementary production enables production of complete or almost complete virus particles that remain self-reproduction limited.

In certain embodiments, the invention relates to a method of production of a virus envelope and/or a fragment of a virus envelope and/or virus envelope protein comprising culturing the host cell of the invention.

The term “virus envelope”, as used herein, refers to protein assembly such as a protein layer that has a stabilizing function for a nucleotide acid sequence (such as the nucleotide acid sequence of the invention). In some embodiments, the virus envelope described herein enables the assimilation of the nucleotide acid sequence of the invention into a human cell. In some embodiments, the virus envelope described herein comprises a spike protein, envelope protein and a membrane protein.

In some embodiments, the invention relates to a fragment of a virus envelope obtainable by gene expression using at least one nucleic acid according to the invention using the vector according to the invention, using the kit according to the invention, or the host cell according to the invention.

The term “fragment of a virus envelope”, as used herein, refers to at least two assembled proteins that form an incomplete virus envelope.

In some embodiments, the invention relates to a virus envelope protein obtainable by gene expression using at least one nucleic acid according to the invention using the vector according to the invention, using the kit according to the invention, or the host cell according to the invention.

The term “virus envelope protein”, as used herein, refers to at least one protein that can form part of a viral envelope.

In some embodiments, the invention relates to a virus envelope, a fragment of a virus envelope and/or virus envelope protein obtainable by gene expression using at least one nucleic acid according to the invention using the vector according to the invention, using the kit according to the invention, or the host cell according to the invention, wherein the virus envelope, the fragment of a virus envelope and/or the virus envelope protein package the at least one nucleic acid according to the invention.

The term “packaged”, as used herein, refers to at least partially engulfed and/or linked. In some embodiments, the packaging nucleic acid of the invention in the virus envelope, the fragment of a virus envelope and/or the virus envelope protein enables entrance into human cells.

The products of the nucleic acid and/or the vector of the invention show a particularly high antigenic similarity to the corresponding functional virus, if the products are embodied in a virus envelope, a fragment of a virus envelope and/or virus envelope protein. Therefore, the elicited/induced immune reaction will likely induce an immune reaction that is particularly beneficial for the actual contact with the functional virus.

The nucleotide acid packaged in the virus envelope, the fragment of a virus envelope and/or the virus envelope protein can be transferred into human cell of a subject and induce production of viral proteins in the human cell. This results in prolonged and enhanced exposure of antigenic virus-like proteins with limited replication capabilities.

Accordingly, the invention is at least in part based on the discovery that the vector described herein enables efficient production of a combination virus-like proteins with limited replication capabilities but similar antigenic effect to the original virus.

In certain embodiments, the invention relates to a kit comprising I.) the nucleic acid sequence of the invention; and II.) at least one SARS-COV-2 sequence part complementary to the nucleic acid sequence comprised in (I.).

In certain embodiments, the invention relates to a kit comprising I.) the vector of the invention; and II.) at least one SARS-COV-2 sequence part complementary to the nucleic acid sequence comprised in the vector in (I.).

In certain embodiments, the invention relates to a kit comprising I.) the host cell of the invention; and II.) at least one SARS-COV-2 sequence part complementary to the nucleic acid sequence comprised in the host cell in (I.).

The inventors found that the nucleic sequences described herein can be used as part of a trans-complementary production system.

Accordingly, the invention is at least in part based on the finding that SARS-COV-2 particles can be produced by trans-complementary methods, as described herein.

In certain embodiments, the invention relates to a virus envelope or a fragment of a virus envelope and/or virus envelope protein, wherein the virus envelope or the fragment of a virus envelope and/or the virus envelope protein a) package the at least one nucleic acid of any one of the invention; and b) are obtainable by gene expression using at least one nucleic acid of any one of the invention.

In certain embodiments, the invention relates to a virus envelope or a fragment of a virus envelope and/or virus envelope protein, wherein the virus envelope or the fragment of a virus envelope and/or the virus envelope protein a) package the at least one nucleic acid of any one of the invention; and b) are obtainable by gene expression using the vector of any one of the invention.

In certain embodiments, the invention relates to a virus envelope or a fragment of a virus envelope and/or virus envelope protein, wherein the virus envelope or the fragment of a virus envelope and/or the virus envelope protein a) package the at least one nucleic acid of any one of the invention; and b) are obtainable by gene expression using the host cell of the invention.

In certain embodiments, the invention relates to a virus envelope or a fragment of a virus envelope and/or virus envelope protein, wherein the virus envelope or the fragment of a virus envelope and/or the virus envelope protein a) package the at least one nucleic acid of any one of the invention; and b) are obtainable by gene expression using the method of the invention.

In certain embodiments, the invention relates to a virus envelope or a fragment of a virus envelope and/or virus envelope protein, wherein the virus envelope or the fragment of a virus envelope and/or the virus envelope protein a) package the at least one nucleic acid of any one of the invention; and b) are obtainable by gene expression using the kit of the invention.

The kit described herein, can be prepared by collecting the necessary host cell(s) and reagents. If the nucleic acids comprised in the kit are present in the form of DNA, it is further preferred that they are present in at least one plasmid, preferably in two or more plasmids. This allows the nucleic acid to be easily introduced into a corresponding host cell, as is also described in the context of the concrete examples below.

In certain embodiments, the invention relates to a pharmaceutical composition comprising a) at least one nucleic acid according to one of the invention; and b) at least one amino acid sequence obtainable by gene expression using at least one nucleic acid of any one of the invention, using the vector of any one of the invention, using the host cell of the invention, using the method of the invention or using the kit of the invention.

The term “pharmaceutical composition”, as used herein, refers to a preparation which is in such form as to permit the biological activity of an active ingredient contained therein to be effective, and which contains no additional components which are unacceptably toxic to a subject to which the formulation would be administered.

In certain embodiments, the invention relates to the pharmaceutical composition of the invention, wherein the at least one amino acid sequence is the virus envelope or a fragment of a virus envelope and/or virus envelope protein of the invention.

In certain embodiments, the invention relates to the pharmaceutical composition according to the invention for use as a medicament.

In certain embodiments, the invention relates to the vector of the invention for use as a medicament.

In certain embodiments, the invention relates to the pharmaceutical composition according to the invention for use in treatment and/or prevention.

The term “treatment” (and grammatical variations thereof such as “treat” or “treating”), as used herein, refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis.

In certain embodiments, the invention relates to the pharmaceutical composition according to the invention for use in the prevention of a SARS-COV-2 infection or at least one symptom thereof.

In certain embodiments, the invention relates to the vector of the invention for use in the prevention of a SARS-COV-2 infection or at least one symptom thereof.

In some embodiments, the symptoms of a SARS-COV-2 infection includes at least one symptom selected from the group of fever, cough, fatigue, difficulty breathing, chills, joint pain, muscle pain, expectoration, sputum production, dyspnea, myalgia, arthralgia, sore throat, headache, nausea, vomiting, diarrhea, sinus pain, stuffy nose, altered and/or reduced sense of smell, altered and/or reduced sense of taste, lack of appetite, loss of weight, stomach pain, conjunctivitis, skin rash, lymphoma, apathy, and somnolence.

In some embodiments the pharmaceutical composition according to the invention for use in the prevention of a SARS-COV-2 infection or at least one symptom thereof is a vaccine.

The term “vaccine”, as used herein, refers to any agent or composition, capable of inducing/eliciting an immune response in a host and which permits to treat and/or prevent an infection and/or a disease. Therefore, non-limiting examples of such agents include proteins, polypeptides, protein/polypeptide fragments, immunogens, antigens, peptide epitopes, epitopes, mixtures of proteins, peptides or epitopes as well as nucleic acids, genes and/or portions of genes (encoding a polypeptide or protein of interest or a fragment thereof).

The term “SARS-COV-2 infection”, as used herein, may also be understood as “COVID-19”.

The structural proteins of coronaviruses have shown to elicit an immune response (see, e.g., Li, J. Y., et al., 2020, Virus research, 286, 198074; Walls, A. C., et al., 2020, Cell, 181(2), 281-292. e6; Chen, Z, et al., 2004, Clinical chemistry, 50(6), 988-995; Peng, Y., et al., 2020, Nature immunology, 21(11), 1336-1345.). The means and methods provided enable to inducing/eliciting an equivalent immune response by the production and administration of a vaccine with the equivalent epitopes and/or particles with reduced immune evading mechanisms. In some embodiments, the vaccine induces production of particles with limited replicative capabilities in subject to

Thus, these vaccines thus differ massively from classical vaccines, which are often derived from animal serum and are therefore molecularly inconsistent. The production from animal organisms is traditionally the method of choice. However, the molecularly unclear products lead to massive quality problems and variation from production batch to production batch. This is also associated with the long approval period and the side effects that are often discovered only late. A molecularly defined product composition, as it can be obtained using the nucleic acid according to the invention, is therefore advantageous.

Furthermore, the vaccine described herein, is both clearly defined and offers a broad range of antigenic epitopes. This results in the advantage that the vaccine has a low or no requirement for adjuvants that enhance the immune response. Such adjuvants that enhance the immune response are typically associated with side effects such as allergic reactions in some patients. Furthermore, the primary active components of the vaccine as described herein are protein-based and are therefore more thermostable compared to other vaccines (e.g., RNA vaccines). The vaccine of the invention is therefore easily transportable and storable due to its stability.

Accordingly, the invention is a least in part based on the discovery, that the vaccine as described herein is particularly useful in the treatment and/or prevention of a SARS-CoV-2 infection.

“a,” “an,” and “the” are used herein to refer to one or to more than one (i.e., to at least one, or to one or more) of the grammatical object of the article.

“or” should be understood to mean either one, both, or any combination thereof of the alternatives.

“and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “include” and “comprise” are used synonymously. “preferably” means one option out of a series of options not excluding other options. “e.g.” means one example without restriction to the mentioned example. By “consisting of” is meant including, and limited to, whatever follows the phrase “consisting of.”

Reference throughout this specification to “one embodiment”, “an embodiment”, “a particular embodiment”, “a related embodiment”, “a certain embodiment”, “an additional embodiment”, “some embodiments”, “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While aspects of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

### BRIEF DESCRIPTION OF FIGURES

FIG. 1: MAP of E-gene plasmid and genes (SEQ ID NO: 27)

FIG. 2: Map of ORF6 plasmid and genes (SEQ ID NO: 28)

FIG. 3: Map of ORF7a plasmid and genes (SEQ ID NO: 29)

FIG. 4: Map of ORF8 plasmid and genes (SEQ ID NO: 30)

FIGS. 5-8: Demonstration of expression of the respective genes in eukaryotic cells (mRNA expression demonstrated by RT-qPCR)

FIG. 5: Expression in hygro-selected Vero cells: E expression (RT-PCR to verify RNA-expression): dark line: amplification with reverse transcriptase (RNA plus DNA), light grey: amplification without RT reaction to demonstrate the level of DNA background (from integrated cellular DNA)

FIG. 6: Expression in hygro-selected Vero cells: orf 6 expression (RT-PCR to verify RNA-expression): dark color: amplification with reverse transcriptase (RNA plus DNA), light grey: amplification without RT reaction to demonstrate the level of DNA background (from integrated cellular DNA)

FIG. 7: Expression in hygro-selected Vero cells: orf 7a expression (RT-PCR to verify RNA-expression): dark color: amplification with reverse transcriptase (RNA plus DNA), light grey: amplification without RT reaction to demonstrate the level of DNA background (from integrated cellular DNA)

FIG. 8: Expression in hygro-selected Vero cells: ORF8 expression (RT-PCR to verify RNA-expression): dark color: amplification with reverse transcriptase (RNA plus DNA), light grey: amplification without RT reaction to demonstrate the level of DNA background (from integrated cellular DNA)

FIG. 9: Demonstration of virus production: Following DNA introduction of the full genome, a virus-typical cytopathic effect is induced

FIG. 10: The virus, after expansion in cell culture, has been titrated and shown to lead to the same virus titers as the clinical reference isolate of SARS-COV-2: the final dilution with infection events is shown with individual plaques in column 3. Row A=uninfected control, Rows B-D=clinical isolate (reference); Rows E-G=rescued virus)

FIG. 11: Map of ORF3a plasmid and genes (SEQ ID NO: 32)

FIG. 12: Map of N-gene plasmid and genes (SEQ ID NO: 31)

FIG. 13: Genomic organization of the 4 DNA fragments, which lead to the intracellular re-constitution of a full-length SARS-COV-2 genome. Inside the transfected target cell, DNA fragments recombine. The initial RNA transcription step is facilitated by a heterologous promoter linked upstream of fragment A along with non-coding signal sequences downstream from fragment D.

FIG. 14: NGS analysis of the faithfulness of 26 virus genomes after cellular reconstitution of functional SARS-COV-2 from four co-transfected DNA segments (SEQ ID NO: 22-24, 68). Upper dark grey bars represent the full-length sequences; single nucleotide changes or deletions are indicated as light bars in the respective sequences (SEQ ID NO: 38-63). The orthogonal marks at the top show the precise positions of the different fragment termini. Lower light grey Bars show the positions and extension of the four DNA fragments A-D.

FIG. 15: A) Cell-free infection of unmodified VeroE6 cells with the same amount of reconstituted virus, either full-length or RVX-13 (comprising SEQ ID NO: 26). B) depicts the viral levels of full-length virus (FL) or the vaccine viruses RVX-13 (comprising SEQ ID NO: 26) and RVX-14 (comprising SEQ ID NO: 65) by quant. RT-PCR in supernatant samples of infected cultures after passage 6 in VeroE6 cells.

FIG. 16: A) Quantitative RT-PCR plot for supernatant samples after six cell-free passages of the full-length reconstituted SARS-COV-2 (detected lines) and of the vaccine viruses RVX-13 (comprising SEQ ID NO: 26) and RVX-14 (comprising SEQ ID NO: 65) (lines below detection threshold). All values for the RVX-vaccine viruses remain below the amplification threshold with no indication of a positive RNA-signal. B) Quantification of a virus standard with a titrated stock of the clinical Wuhan isolate. Virus amount from left: 3×10e7; 3×10e6; 3×10e5; 3×10e4; 3×10e3.

FIG. 17: Plasmid map for the M-plasmid (pcDNA3.1hygro(+) _M (SEQ ID NO: 64)).

### EXAMPLES

### Example 1—Reconstitution of the Viral Genome

The SARS-COV-2 genome described in this application is produced in the form of 1-8 complementing segments, which reconstitute the complete viral genome with all genes of SARS-COV-2 or a viral genome with all genes of SARS-COV-2 except for the ones that were deliberately eliminated, (i.e. E-gene, orf 6, orf 7a, orf 8). For initiation of the production of the viral RNA genome, a separate promoter element, e.g. from Cytomegalovirus, is attached to the 5′ end of the genome; the 3′ end is engineered to contain a poly A tail of suitable length, a ribozyme cleavage element and a eukaryotic poly A signal (e.g. SV40 or bGH). The final 1-8 segments can be re-assembled in two principal ways:
1 either prior to introduction into the cell, using published methods such as Gibson assembly or site-specific ligation using a ligase enzyme to connect type II restriction sites, which had been engineered to the termini of each fragment. Of note, for the introduction of restriction sites, the alteration of the protein was avoided or minimized (limited to conservative changes), or
2 by engineering the 1-8 fragments in a way that the termini of adjacent fragments possess a sequence overlap (identical sequence in both adjacent fragments) of 30-40nucleotide pairs. With appropriate means these fragments are then introduced in stoichiometric amounts into the target cell, in which the reassembly of the complete SARS-COV-2 genome will occur via recombination, facilitated by cellular enzymes.

A further alternative is the introduction of extracellularly in vitro produced RNA of the entire SARS-COV-2 genome, or a viral genome with all genes of SARS-COV-2 except for the ones that were deliberately eliminated, (i.e. E-gene, orf 6, orf 7a, orf 8), which can be obtained by linking a T7 promoter to the 5′ end of the viral genome. Commercial T7 polymerase allows the efficient production of genomic SARS-COV-2 RNA, which can be introduced by published means (e.g. electroporation or transfection reagents such as Jet-messenger etc.).

### Example 2—Cellular Introduction

The cell lines used for this process are preferably HEK293 cells but can be other cells suitable for effective DNA introduction by transfection, such as HeLa, BHK, or Vero-clones.

For the efficient introduction, special commercially available facilitators are used, preferentially Lipofectamine 3000 or jetPRIME, but also other related products or methods using Ca-Phosphate or electroporation. For this process, manufacturers' protocols or adaptations of the same are used.

Methods for the coexpression of the necessary complementing genes or of viral genes, which are needed for efficient vaccine production (e.g. expression plasmids or RNA of the viral nucleocapsid gene) are co-transfected with the genomic nucleic acid.

Since the introduced viral vaccine genomes miss defined genes of SARS-COV-2, those gene products have to be provided either by the host cell (stable transduced or transfected before-hand) or by co-transfection of expression plasmids for the missing genes.

### Example 3—Virus Recovery

After introduction of the nucleic acid constructs into the target cells, the production of viral RNA templates is initiated spontaneously, either from the introduced RNA genomes or after transcription of the DNA genome. Mechanistically, negative-stranded RNA genomes are produced, which then serve as template for the positive stranded mRNAs and the genomic full-length RNA.

Since the expression in such a transient introduction situation by transfection declines after 3-4 days, the transfected cultures are co-cultivated with susceptible cells, i.e. those cells, which constitutively express the missing genes. As a result, the transfected cells will transmit the virus progeny directly to the second cell type of cells, expressing the missing genes. In these latter cells, a continuous infection is initiated, leading to the production and release of free virus particles.

These particles will now be fully infectious only for the cell line expressing the missing genes (producer cell), allowing the propagation of the vaccine virus. In contrast, when these virus particles are used to infect naïve cells (with the complementing functions missing), no viral replication will occur.

### Example 4

This cell system reflects a biologically safe system for the production of single-cycle virus, which is only infectious as long as the producer cell is used. Due to this restriction, the virus production can be transferred to a lower biosafety level 2. This facilitates the easy use of this system for diagnostic purposes: Instead of requiring the biosafety level 3 for SARS-COV-2, the engineered cells plus deleted virus genomes can be handled in standard diagnostic settings.

Yet, since the complementation allows virus propagation (restricted to the very cell and very virus type), plaque reduction assays and virus neutralisation tests can be performed using this invention.

### Example 5

### Versatility of the Clonal System

We foresee a great versatility of the “cassette system”, employing the molecular reconstitution of a deletion-carrying virus genome utilizing up to 8 subgenomic fragment by the technical flexibility to rapidly introduce relevant mutations and alterations into specific target genes, which are found in only 1 of the fragments. As example, the S-gene, present only in fragment 7 (of 8 fragments) or in Fragment 4a (4 fragments), can readily be manipulated in vitro and re-introduced into the genomic assembly without any need for manipulating any of the other gene segments.

With this step, the process will be able to easily address viral variation (as seen in the currently emerging variants of clinical concern) and, at the same time, retains the perfect sequence of all other genomic regions.

### Example 6

On day 7 after transfection and culture in a susceptible Vero cell line, cytopathic changes lead to the production of virus plaques in the cell layer.

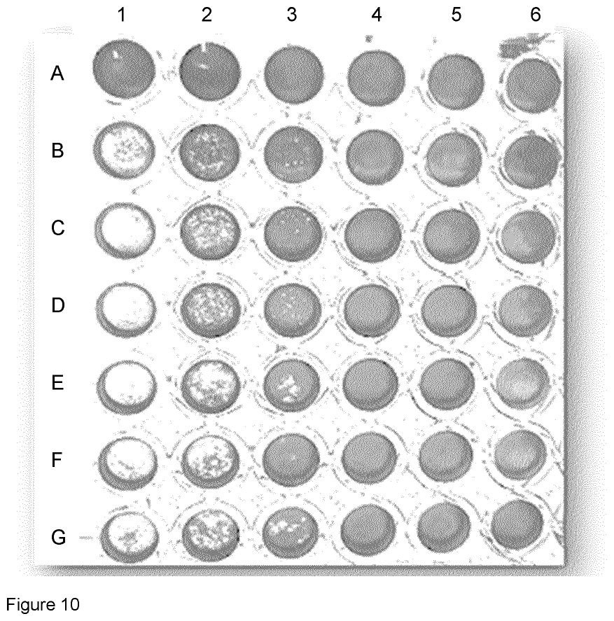

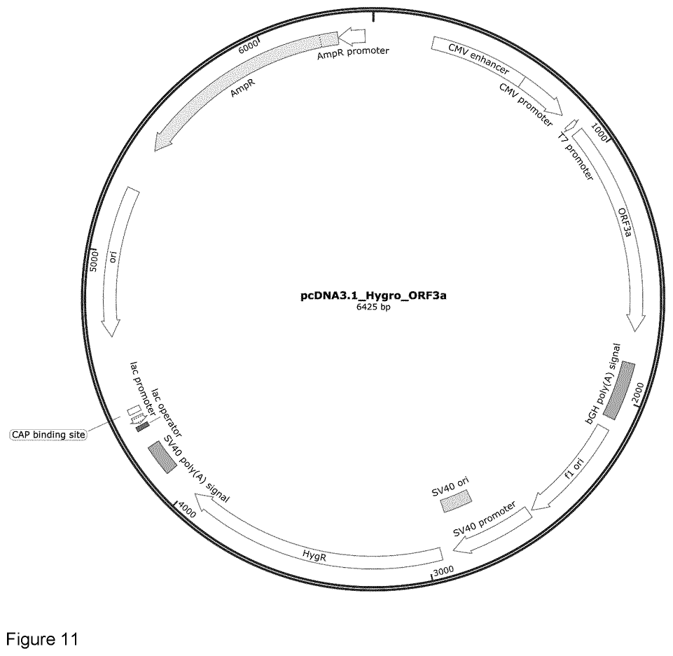

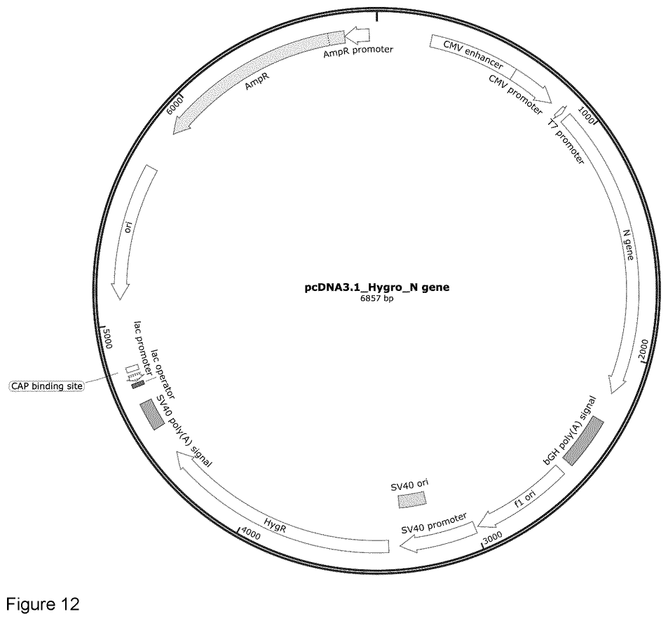

Virus titration (FIG. 10) was performed by a serial 2-fold dilution of each virus stock. After plating onto susceptible Vero cells and 48 hrs of incubation, cultures were fixed, stained with crystal violet and microscopically inspected for viral plaque

### Materials and Methods

Sequence verification of the viral genome and of the presence of genes in the producer cell
Cell establishment, selection process

Expression plasmids of the expressible, isolated viral genes utilize either standard expression vectors or constructs, in which inducible promoters
Verification of expression

After stable introduction and expansion of cell clones surviving the antibiotic selection step, mRNA expression is demonstrated, and for some genes also protein expression.
Transfection protocol

Cells are transfected with a suitable DNA-or RNA transfection method, using lipid-based facilitating reagents, Ca-phosphate or electroporation using standard protocols or adaptations thereof.

After cell culture and/or cocultivation of transfected transient expressor cells (293T, BHK) with susceptible producer cells (Vero+E+7, etc.), virus production could be demonstrated by the spontaneous occurrence of a coronavirus-typical cytopathic effect, by RT-PCR of filtered supernatant for the titer of viral RNA, and via plaque assay using stepwise dilutions of 1st generation viral supernatant on susceptible producer cells
Functional complementation protocol: proof of virus production

Transfection of producer cells with the virus deletion variant is followed by an extended culture period, during which the spontaneous development of cytopathic changes (CPE), i.e. plaque formation is monitored by microscopic inspection. As soon as increasing CPE and cell death is noted, cell-free supernatant samples are transferred onto a layer of uninfected producer cells. The development of CPE after about 2 days and the simultaneous demonstration of SARS-COV-2 specific RNA by RT-PCR serve as proof of viral replication.
Infection protocol and read-out

Susceptible cells were incubated with dilutions of vaccine virus using inoculum titers of 0.1 to 0.01. From day 2 after infection, cell viability and plaque-formation were inspected, and virus harvested on days 3-5.
Virus propagation, stock production

Virus supernatant from infected cultures was obtained by removal of culture supernatant and clarification by centrifugation. Virus aliquots were stored frozen at −70° C., and virus titers determined in a standard plaque assay using susceptible producer cells. For testing, infected cells were overlayed with low-melting agarose, fixed on day 2 and stained with crystal violet for enumeration of infection events (=plaques)

### Example 7

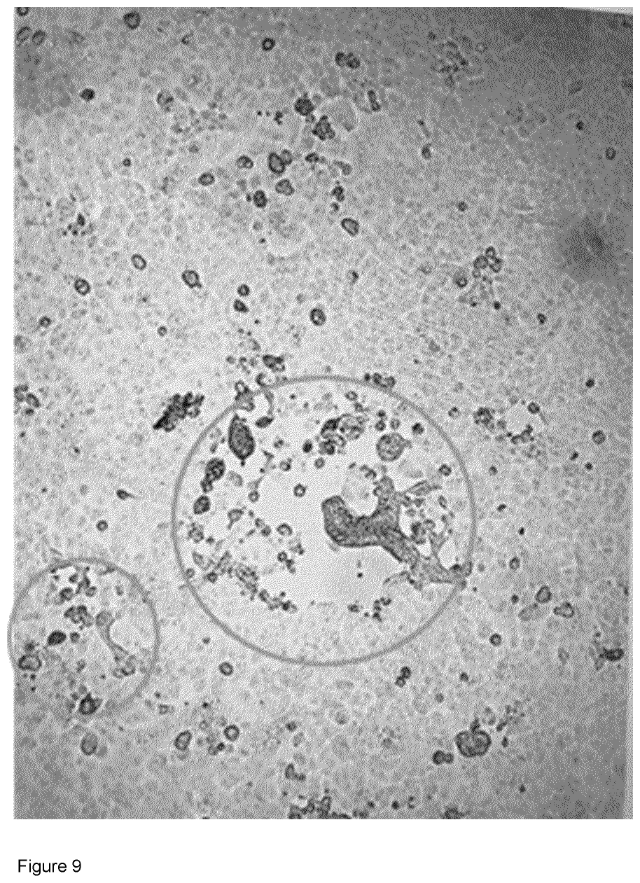

1) The complete SARS-COV-2 genome, flanked by the cytomegalovirus promoter (CMV) at the 5′ and a poly A tail of 30-35nt length, the hepatitis delta ribozyme and simian virus 40 polyadenylation signal (HDV/SV40) at the 3′ termini, was cloned into four plasmids and PCR amplified with Q5 high-fidelity polymerase (M0491S, NEB). For this approach, the complete viral genome including all genes was amplified to generate wild type virus for proof of principle. PCR primers were designed to generate 20-25 nt overlaps between the adjacent fragments to enable subsequent assembly of the full-length genome by the Gibson method (Gibson, et al., 2009, Nature Methods 6 (5): 343-345). The NEBuilder HiFi DNA Assembly cloning kit (E5520S, NEB) was used and the manufacturers protocol was followed. Without purification, this product served as template for another round of PCR to further amplify the full-length product. Following EtOH purification, 2 ug of the full-length viral DNA genome were transfected into 4×10\hat{ }5 293T cells using jetPRIME (114-07, Polyplus). The next day, susceptible Vero E6/TMPRSS2 cells were added at 30% confluency. Supernatant from this co-culture was passaged onto fresh Vero E6/TMPRSS2 cells and first CPE was detected eight days post transfection. Presence of infectious virus was confirmed by passaging the supernatant twice onto fresh Vero E6/TMPRSS2 cells and confirming CPE (FIG. 9), RT-qPCR and NGS sequencing of the supernatant. The latter confirmed the presence of an unique Sall site that was introduced by silent mutation for identification.

2) A second strategy follows the ISA (infectious subgenomic amplicons) method described by Aubry et al. 2014 The Journal of General Virology 95 (Pt 11): 2462-2467. Transfection of overlapping double-stranded DNA fragments will lead to a full-length viral DNA copy after intracellular recombination. In this approach, four fragments were amplified from the plasmids described before (frA, frB, frC, frD) with primers designed to generate 100 nt homology regions between the fragments. Amplicons were purified using the QIAquick PCR purification kit (28104, Qiagen) and 2.5 ug of an equimolar mix was transfected into 4×10\hat{ }5 293T cells using Lipofectamine-3000 (L3000001, Invitrogen). After transfection, the same procedure was carried out as described in 1.

The first as well as the second strategy worked on the in trans complementing cell lines, proofing the capability of these cells to be transfectable as well as infectable. For the production of virus missing the eliminated genes, fragment D will be replaced by fragment D1 (SEQ ID NO: 25) or D2 (SEQ ID NO: 26) and only cell lines expressing the eliminated genes in trans will be used. The same protocols as described in 1) and 2) will be followed as well as the following:

3) A small sequence inserted between the CMV promoter and the 5′ UTR encodes for the T7 promoter which enables the in vitro transcription of genomic full-length mRNA. Following the published work by Xie and colleagues with minor changes, the RiboMAX Large Scale RNA production system (P1300, Promega) was used for production of viral mRNA (Xie et al., 2021, Nature Protocols 16 (3): 1761-1784). In short: 20 ug of full-length mRNA together with 10 ug of N mRNA were electroporated into 1×10\hat{ }6 Vero E6/TMPRSS2 cells using the Amaxa 4D nucleofector device (Lonza), following the manufacturers protocol.

4) The four fragments covering the whole SARS-COV-2 genome, except the deliberately eliminated genes, were designed to have typellS restriction sites on their 5′ and 3′ ends for liberating the fragments from the plasmid backbone. After digestion with the corresponding enzyme specific error-free ligation of the full-length viral DNA genome can be achieved using T4 DNA ligase (M0202S, NEB). The product will be purified using the QiaEX II Gel Extraction kit (20021, Qiagen) or EtOH precipitation. This 32kb DNA construct will be transfected using a suitable transfection reagent (jetPRIME, Lipofectamine-3000, Lipofectamine-LTX) or electroporated using the Amaxa 4D nucleofector device (Lonza).

### Example 8

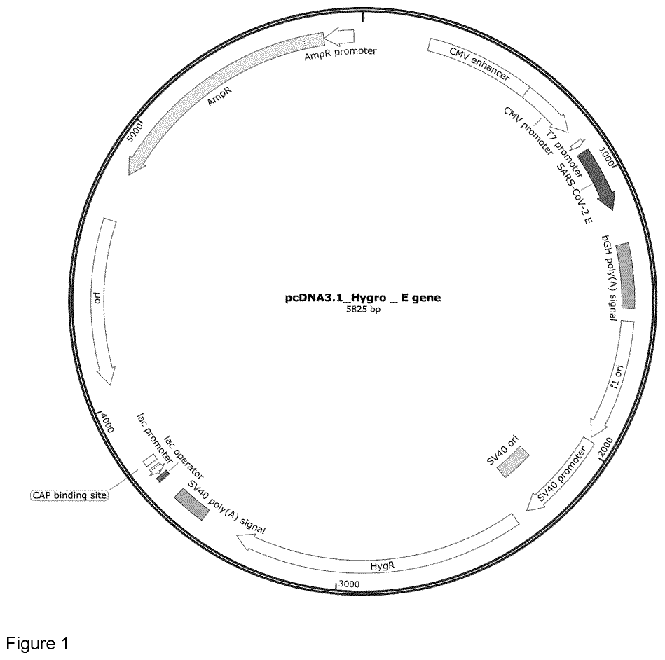

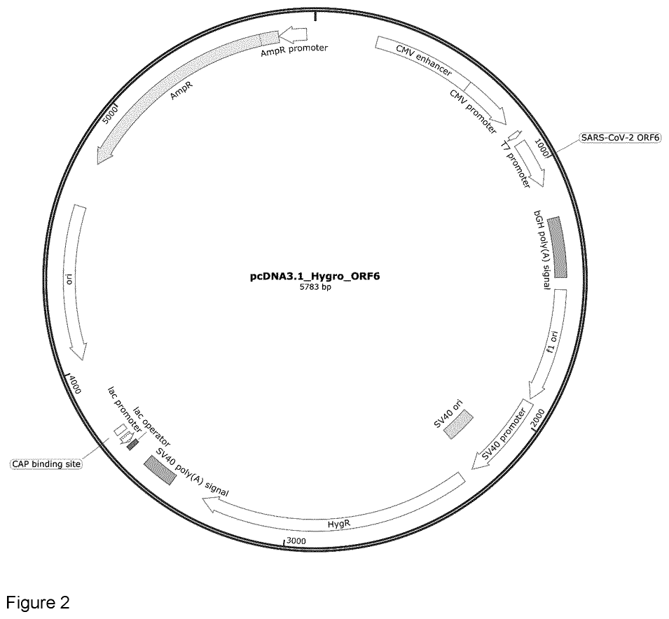

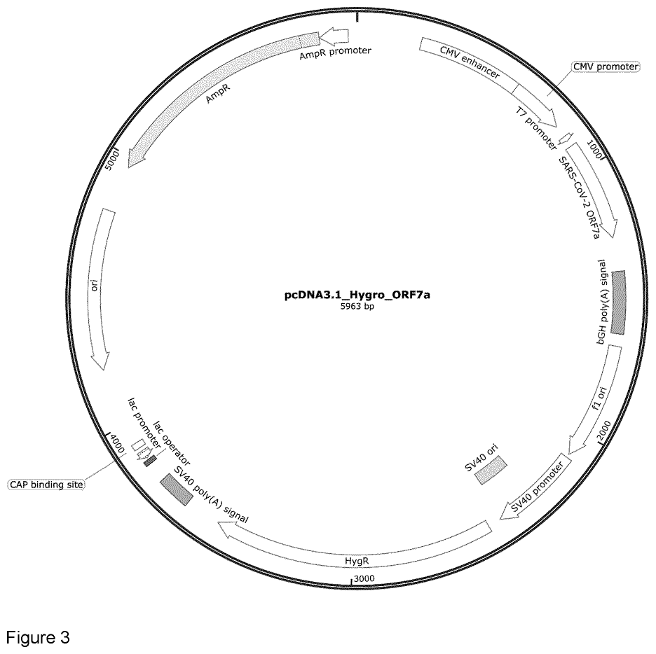

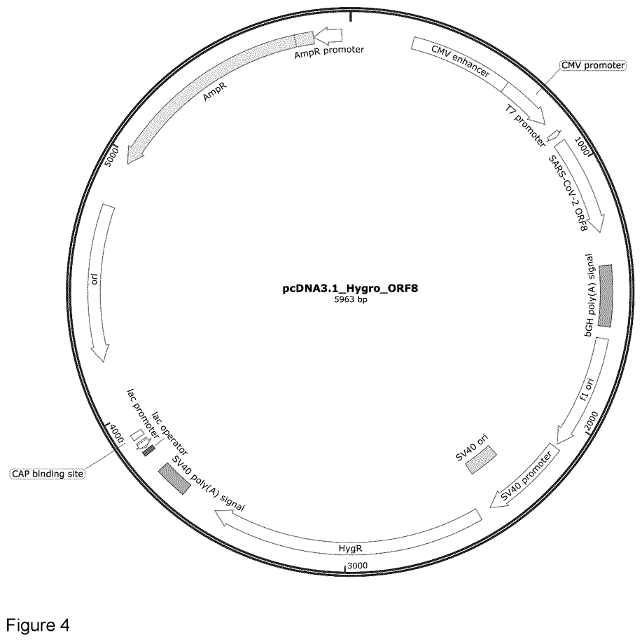

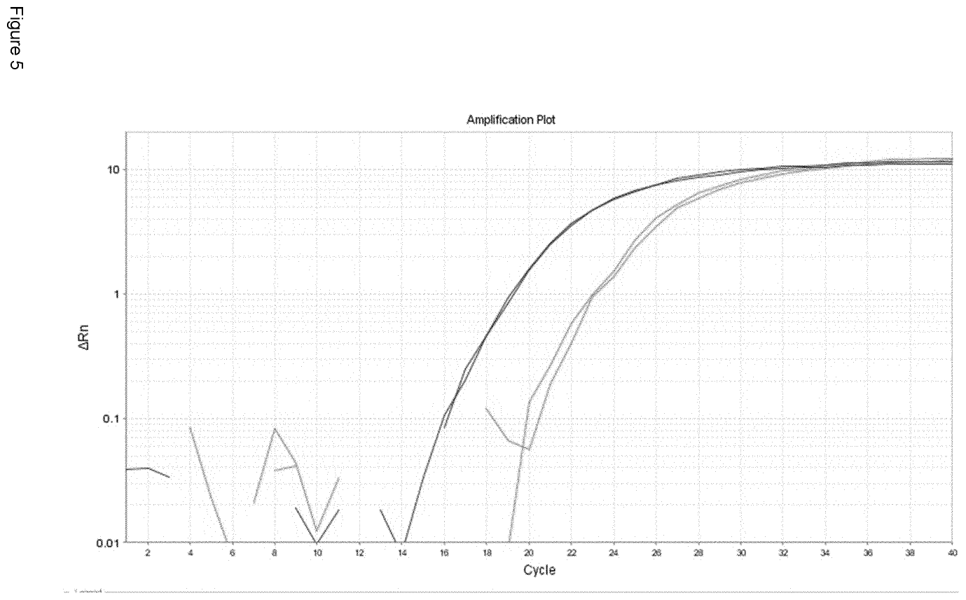

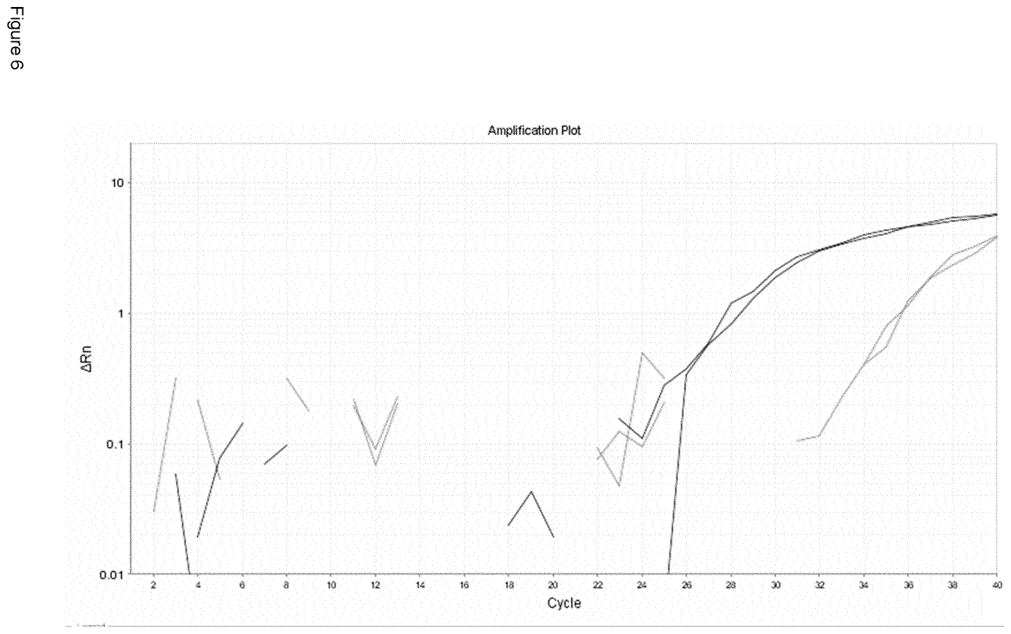

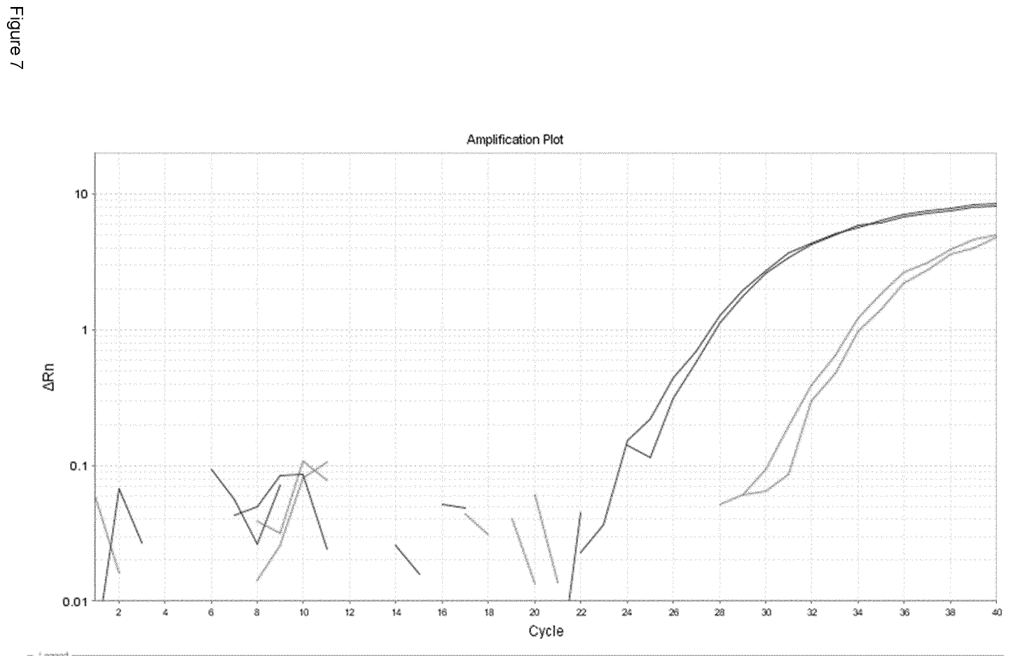

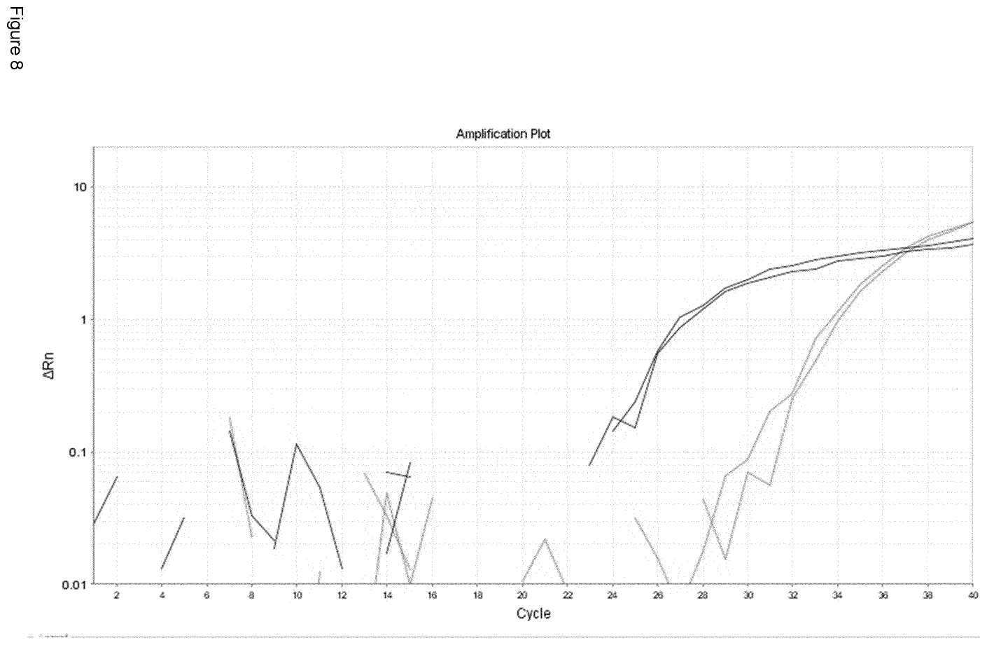

Single-stranded RNA corresponding to the vaccine virus genome was obtained by in vitro transcription using T7 polymerase. The so obtained RNA was transfected into suitable cell lines (HEK293T or Vero cells). In the case of the positive control, the full-length construct, unaltered HEK293 or Vero cells supported the replication of the RNA genome, the generation of subgenomic mRNAs and hence translation into viral proteins. These, together with the positive-strand RNA genome, and components from the cell membrane, formed progeny viruses, in this case wild-type, natural SARS-COV-2 viruses. In the case of the deletion mutants, the gene or genes deleted in the virus genome are transfected into the cell lines in the form of DNA (see FIGS. 1-4), leading to the transient expression of the protein or proteins, and thereby providing the missing factor required for enabling the generation of progeny virus. Alternatively (and preferred), cultivation of those cells under selection pressure leads to the stable integration of the gene or the genes into the cell genome, from where the protein or proteins are continuously expressed (with expression we understand the generation of mRNA from the gene (see FIGS. 1-4) and the subsequent translation into proteins). Such cells, either transiently or stably expressing the proteins made from the genes missing in the vaccine virus genome, enable a continuous production of vaccine viruses, characterized by a full set of structural proteins and a vaccine virus genome having one or several genes deleted. The so obtained vaccine viruses were purified in a so-called downstream processing (DSP) process characterized by clarification (separation of cells from the vaccine viruses), DNA digestion by Benzoase, Ultra Filtration/Dia Filtration (“UF/DF”) and finally sterile filtration (0.22 um filtration).

### Example 9

### Demonstration of Biological Safety of RVX-13, RVX-14

The inventors took the approach to utilize the precise excision of the coding information for the E-gene alone (RVX-14 comprising SEQ ID NO: 65) or in conjunction with 1-2 additional genes, which are responsible for the cellular immune defense (RVX-13 comprising SEQ ID NO: 26). The missing function will then be supplied (=transcomplemented) via a special “producer cell” and never appear in the genome of the viral vaccine.

The SARS-COV-2 vaccine candidate RVX-13 (comprising SEQ ID NO: 26) and RVX-14 (comprising SEQ ID NO: 65) and as well as the candidate MoVi-1 (comprising SEQ ID NO: 36) represent unique and completely “cycle-blocked” vaccine viruses, unable to replicate.

Specifically, RVX-13 was assembled from fragments A, B, C, D and D2 (SEQ ID NO: 22, SEQ ID NO: 23, SEQ ID NO: 24, SEQ ID NO: 26 and SEQ ID NO: 68) according to the methods of the previous examples.

RVX-14 comprises the sequence as defined by SEQ ID NO: 65 and is assembled from fragments A, B, C (SEQ ID NO: 22, SEQ ID NO: 23, SEQ ID NO: 24) and D14 (D14 is a sequence constructed based on a fragment D2 (SEQ ID NO: 68) amended such that a sequence as defined by SEQ ID NO: 65 is located between the sequence part encoding the SARS-COV-2 N protein and the sequence part encoding ORF3a) according to the methods of the previous examples.

MoVi-1 comprises the sequence as defined by SEQ ID NO: 36 and is assembled from fragments A, B, C (SEQ ID NO: 22, SEQ ID NO: 23, SEQ ID NO: 24) and Dv1 (Dvi is a sequence constructed based on a fragment D2 (SEQ ID NO: 68) amended such that a sequence as defined by SEQ ID NO: 36 is located between the sequence part encoding the SARS-COV-2 N protein and the sequence part encoding ORF3a) according to the methods of the previous examples.

The basis for this is that the viral genome is missing the described critical gene(s) that are essential for viral replication in normal cell lines susceptible to SARS-COV-2 infection.

For producing the respective inactive candidate vaccines, special genetically modified cell lines have been designed, which constitutively produce the missing viral function. As a consequence, the infection of these manipulated SARS-COV-2-susceptible cells with the “cycle-blocked” vaccine viruses leads to a “trans-complementation” of the genetic information missing in the incoming viral genome with the viral proteins already produced in this special cell line.

As a result, the defective viruses will incorporate the viral protein, which is provided by the producer cell, and are now able to produce functionally genuine particles that, however, continue to contain only defective viral RNA genomes.

This trans-complementation of SARS-COV-2 by a “cell-based viral gene” is a very safe process and does not lead to DNA-recombination in the producer cell, since the transgene DNA exclusively localizes to the cellular nucleus, while the replication of SARS-COV-2 as a positive-stranded RNA virus is confined to the cytoplasmic compartment.

In the following, evidence to prove safety and stability of the proposed viral production system is provided that is completely replication-blocked in any unmodified normal cell line, susceptible to wild-type SARS-COV-2 infection.

Consequently, the inventors provide the experimental proof required to allow lowering the biosafety level for the cycle-blocked vaccine viruses RVX-13 (comprising SEQ ID NO: 26), RVX-14 (comprising SEQ ID NO: 65) and MoVi-1 (comprising SEQ ID NO: 36) to biosafety level BSL-2.

### The Vaccine Virus is Faithfully Reconstituted by an Intracellular DNA Recombination Step

For virus reconstitution the inventors utilize four DNA segments, which overlap by 100 bp, and allow the functional restoration of a full-length viral genome, which then carries the desired deletions. This initial reconstitution represents a necessary first step of the vaccine production process.

For validation, multiple independent reconstitution experiments were conducted, in which all four subgenomic DNA fragments, necessary to re-generate the complete viral genome, were simultaneously introduced into the susceptible target cell lines HEK293 or VeroE6. The intracellular recombination and repair into full length viral genome happens by a cell-driven spontaneous process, and was assessed by analyzing the emergence of infectious virus progeny. Emerging virus in the cell-free culture supernatant was analyzed by NGS, demonstrating the reproducible repair in a highly defined manner:

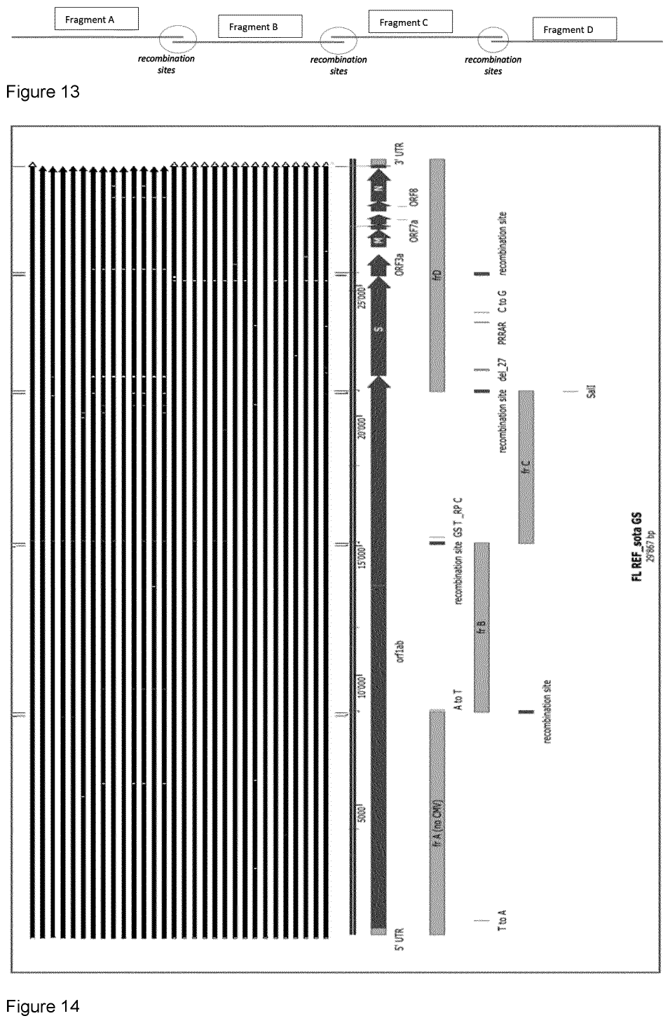

The inventors demonstrate that each recombination and ligation step between the four fragments sketched in FIG. 13 is occurring in a highly faithful manner, as shown by sequence analysis in the emerging virus product at each of the three junctions in 26 independent reconstitution experiments. This is summarized in FIG. 14.

An in-depth NGS sequence analysis at 1 and 10% cutoff revealed only very few sequence differences from the reference DNA, a clinical Wuhan isolate of SARS-COV-2, which was the starting point for cloning. I.e., none of the analyzed genomes had more than 9 mostly silent or conservative point mutations to the reference in their 30000 nucleotide genome lengths, and no single change was noted, mapping to the recombination region at the fragment junctions.

These data confirm that the SARS-COV-2 genome recombination by the ‘IDRA-technique’ (for ‘Intracellular DNA-Recombination and Assembly”) to generate the vaccine virus candidates RVX-13 (comprising SEQ ID NO: 26), RVX-14 (comprising SEQ ID NO: 65) and MoVi-1 (comprising SEQ ID NO: 36) is highly precise.

### Genetic Stability of the Vaccine Virus During Replication

The deletion-carrying vaccine virus is to be added to a production cell line containing the missing structural gene(s) as transgenes. Only by means of this unique property of the producer cell, the vaccine viruses can be replicated. To demonstrate (i) a precise reconstitution, (ii) the absence of aberrant gene recombination and (iii) stability during virus production, the viral genomes from such newly formed virions of the vaccine viruses were analyzed by NGS.

To exclude one-off events, infections of the production cells and subsequent NGS analysis were conducted several times, independently of each other. In summary: After infection and about 5-10 virus generations, no selection of any consistent mutational patterns or deletions in relevant genes was observed.

In the 26 analyzed virus reconstitutions, maximally 9 SNPs with mostly silent mutations were observed, indicated by light marks in the top panel of FIG. 14.

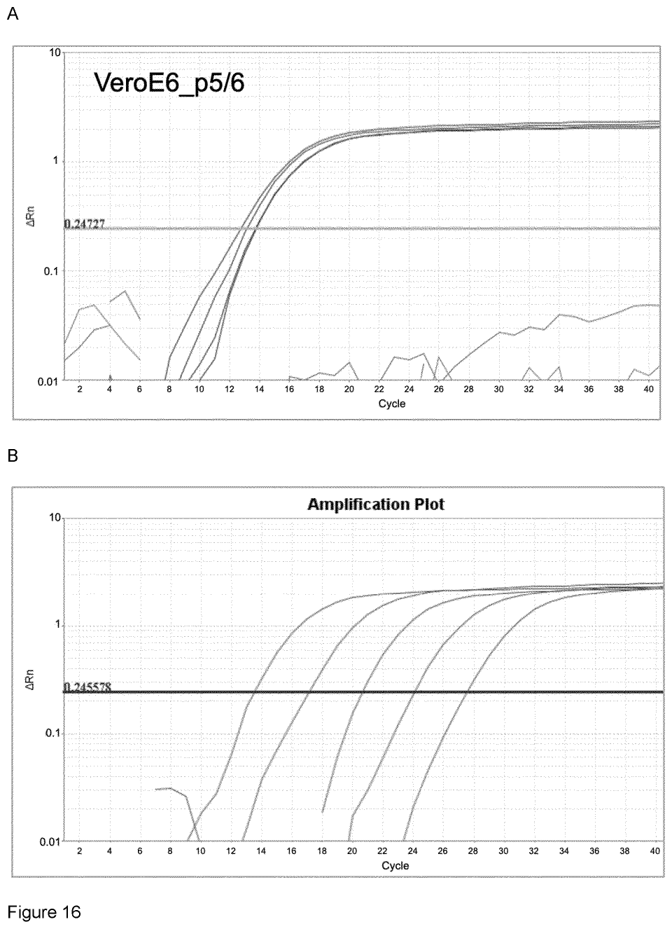

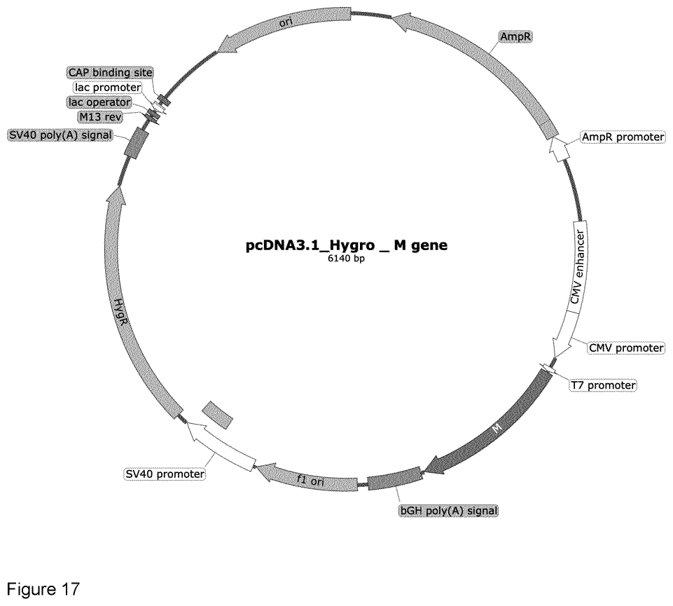

The detailed analysis of all re-joined fragment junctions was conducted with the NGS information for all reconstituted and replication-competent virus isolates. It revealed a high fidelity and the absence of mutations for all of the three junctions, as shown in the example for fragments B and C in FIG. 16.

The very high sequence-identity between all isolated virus sequences is compiled in FIG. 14. This demonstrates the high fidelity of the intracellular DNA repair mechanism, which leads to the re-composition of fully functional SARS-COV-2 genomes.

These data prove that the vaccine virus faithfully replicates in a highly reproducible manner without genetic alterations, virus sequence adaptations, or recombination with cellular genes.

Proof that the Vaccine Viruses Cannot Replicate in Unmodified VeroE6 Cells

It is of utmost importance to verify that the vaccine viruses RVX-13 (comprising SEQ ID NO: 26), RVX-14 (comprising SEQ ID NO: 65) and MoVi-1 (comprising SEQ ID NO: 36) cannot replicate in cell lines, which are typically fully susceptible to SARS-COV-2 replication, such as VeroE6 or 293 HEK cells, expressing the human ACE-2 and TMPRSS2 proteins for viral entry.

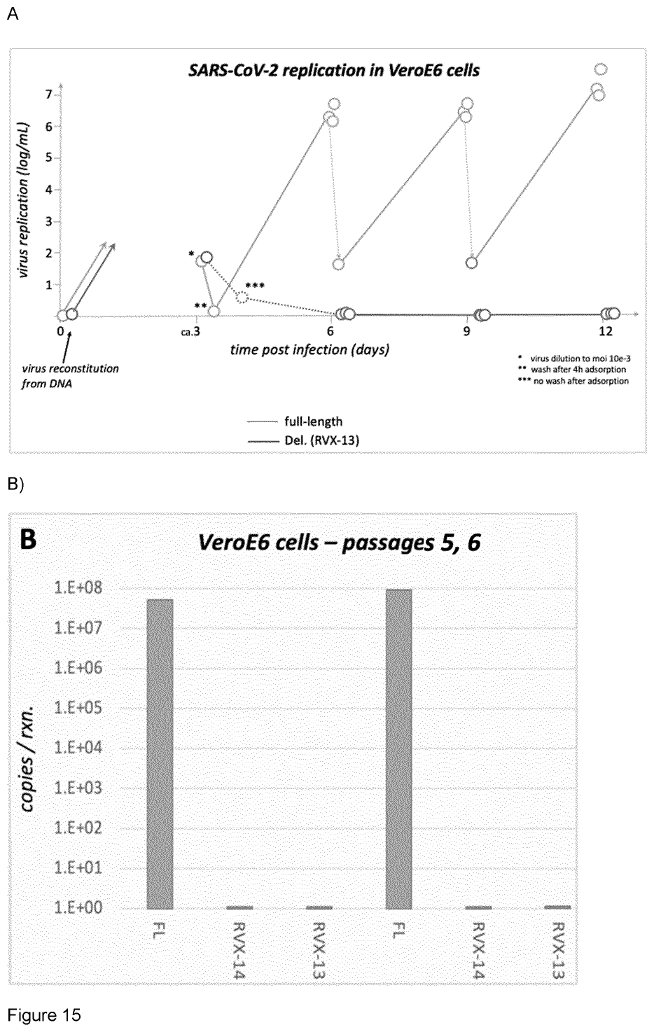

Experimental details of the culture infections leading to the data shown in FIG. 15: After viral reconstitution from DNA on day 0, emerging virus particles were harvested from the culture supernatant (after detection of N-protein in culture supernatant around day 3) and filtered. Virus inoculation in 10 separate parallel infection experiments was done on day 3 at a multiplicity of infection (moi) of ca. 10⁻³, to allow maximal virus spread and propagation). For infection, virus was adsorbed for 4 hrs. After the adsorption period, and to ensure a maximal stringency, the culture medium containing full-length virus (blue line) was completely removed and cells washed 3 times with PBS (**). As the replication competence of the vaccine virus was expected to be lower, the inoculum of RVX-13 (comprising SEQ ID NO: 26) was left on the cultures. Medium was changed only one day later with no washing (***). Infected cultures were continued for 2-3 days to allow maximal virus propagation. Then, supernatant was sampled and analyzed for virus.

The harvested FL virus was diluted again to a multiplicity of one infectious unit per 1000 cells (a moi of 10⁻³) to initiate a new infection. This procedure will enable us to follow any genetic evolution and adaptation occurring during multiple infection rounds. In order to compensate for an assumed lower infectivity of the vaccine virus, the RVX-13 vaccine virus (comprising SEQ ID NO: 26), a larger volume of 1/10 of the harvested culture supernatant was added as “putative inoculum” to fresh uninfected VeroE6 cells at each blind passage.

The continuous logarithmic amplification of the reconstituted full-length virus (FL) confirms a full susceptibility of the cell line to infection, and a similar replication pattern of full-length virus is seen after infection of VeroE2T cells, which provide the missing gene.

In sharp contrast, a complete absence of virus propagation was observed already after the first virus passage for the vaccine viruses RVX-13 (comprising SEQ ID NO: 26) and RVX-14 (comprising SEQ ID NO: 65).

To exclude one-off events, the addition of the vaccine virus to unmodified cells was repeated, and analyzed by quantitative RT-PCR several times, independently of each other (FIG. 16). The complete absence of signal demonstrates for several independent experiments that the vaccine virus does not produce progeny virus and that it cannot spontaneously revert in unmodified cells in such a way that an infectious, replication-capable wild-type or wild-type-like SARS-COV-2 would emerge.

Sequential virus passaging has been carried on to 6 such passages of either full-length virus or the two vaccine virus candidates RVX-13 (comprising SEQ ID NO: 26) and RVX-14 (comprising SEQ ID NO: 65).

While full-length virus continues to yield very high titers within 2-3 days (quantified by RT-PCR: Ct value of ca. 12, FIG. 16), none of the passaged vaccine virus candidates RVX-13 (comprising SEQ ID NO: 26) or RVX-14 (comprising SEQ ID NO: 65) show any sign of virus propagation, even when amplified to 40 PCR cycles (no Ct value). Already at first passage no infectious vaccine virus was detectable in the culture media.

The quantitative RT-PCR protocol used for our experiments is targeting a viral gene that is not affected by any of the mutations introduced to generate RVX-13 (comprising SEQ ID NO: 26) or-14 (comprising SEQ ID NO: 65). This quantitative in-house protocol has been validated against an official diagnostic protocol (Corman et al, Euro Surveill.2020: 25 (3); doi: 10.2807/1560-7917. ES.2020.25.3.2000045).

The complete absence of viral replication of RVX-13 (comprising SEQ ID NO: 26) and RVX-14 (comprising SEQ ID NO: 65) over multiple passages in standard cell lines used for SARS-COV-2 propagation (VeroE6, HEK293-TA) proves the biological safety of the vaccine virus;

This justifies to apply a lower biosafety level for work with these single-cycle vaccine viruses;

The simultaneous ability to grow virus from the same stocks in specialized producer cells; which provide the missing viral gene(s) renders the vaccine viruses producible for further use.

### Absence of Reversion or Virus Evolution in Vitro; Lack of Recombination Between Viral Genomic RNA and Transgene

The extensive molecular data package shown in FIGS. 14- 16 strongly supports the statement that the disabled vaccine viruses RVX-13 (comprising SEQ ID NO: 26) and RVX-14 (comprising SEQ ID NO: 65) cannot undergo molecular changes, which would facilitate the restoration of replicative virus.

Furthermore, one remote theoretical concern could be that the viral RNA genome might find a way to recombine with the complementing transgene, which is present in the nucleus of the producer cell. This repair step should then lead to the re-generation of a virus genome, which is similar to the full-length control used in our experiments.

However, such an event with emerging full-length virus (with its superior replication capacity) has never been observed, and also our extensive sequence analyses of infections by NGS did not reveal any hint for such recombination event between the cytoplasmic viral genomes and cellular DNA information.

In the experimental setup delineated above, the complete absence of any viral recovery in vitro after multiple sequential virus passages serves as solid evidence that no viral reversion to “wild-type” was and will be possible during in vitro passage.

This finding fully supports the intention of the molecular design strategy for the vaccine viruses RVX-13 (comprising SEQ ID NO: 26) and RVX-14 (comprising SEQ ID NO: 65) or MoVi-1 (comprising SEQ ID NO: 36): The complete open reading frame of the viral genes of interest was deleted, leading to a situation that does not allow any recombination of “residual sequences” with any counterpart in the producer cell, thus eliminating viral genome repair.

### Summary

1. The inventors have demonstrated that, after multiple cell passages of vaccine viruses RVX-13 (comprising SEQ ID NO: 26) and RVX-14 (comprising SEQ ID NO: 65) in a genetically engineered producer cell, more than 99% of its original, authentic sequence is fully retained after the 6th generation of the vaccine viruses (further passaging is continued).
2. The inventors show that in ten parallel infections of the vaccine viruses RVX-13 (comprising SEQ ID NO: 26) and RVX-14 (comprising SEQ ID NO: 65) and after multiple blind-passages on VeroE6 cells, no viable virus emerges; already after the first passage no replicative virus can be demonstrated in normal, SARS-COV-2susceptible cells.
3. A highly sensitive analysis of RVX-13 (comprising SEQ ID NO: 26) and RVX-14 (comprising SEQ ID NO: 65) by next-generation sequencing (NGS) reveals that after sequential passages in permissive producer cells, the population of offspring vaccine virus is found to be well conserved, containing less than 0.1% codon-changing point mutations.
4. This demonstrates that the vaccine virus cannot and does not spontaneously change during virus propagation in cell culture in the producer cells and remains unable to re-generate infectious, replication-competent wild-type or wild-type-like SARS-COV-

1. A biologically produced nucleic acid sequence comprising
a) two or three primary nucleic acid sequence parts, wherein a primary nucleic acid sequence part encodes an amino acid sequence selected from the group consisting of
i) SEQ ID NO: 1 (SARS-COV-2 N) or an amino acid sequence with at least 90% sequence identity thereof;
ii) SEQ ID NO: 2 (SARS-COV-2 S) or an amino acid sequence with at least 90% sequence identity thereof;
iii) SEQ ID NO: 3 (SARS-COV-2 E) or an amino acid sequence with at least 90% sequence identity thereof; and
iv) SEQ ID NO: 4 (SARS-COV-2 M) or an amino acid sequence with at least 90% sequence identity thereof; and
b) not more than three, not more than two not more than one or no secondary nucleic acid sequence part(s), wherein a secondary nucleic acid sequence part encodes an amino acid sequence having the function of a SARS-COV-2 amino acid sequence encoded by ORF3a, ORF6, ORF7a, or ORF8, wherein if no sequence part of a) iii) and no nucleic acid sequence part that encodes an amino acid sequence having the function of a SARS-CoV-2 amino acid sequence encoded by ORF3a are present, then not more than five, not more than four, not more than three nucleic acid sequence parts selected from a)i), a)ii), a)iv), and nucleic acid sequence parts encoding an amino acid sequence having the function of a SARS-COV-2 amino acid sequence encoded by ORF6, ORF7a, or ORF8 are present.

2. The nucleic acid sequence of claim 1, wherein the nucleic acid sequence comprises two or three primary nucleic acid sequence parts, wherein a primary nucleic acid sequence part encodes an amino acid sequence selected from the group consisting of
i) SEQ ID NO: 1 (SARS-COV-2 N) or an amino acid sequence with at least 90% sequence identity thereof;
ii) SEQ ID NO: 2 (SARS-COV-2 S) or an amino acid sequence with at least 90% sequence identity thereof; and
iii) SEQ ID NO: 3 (SARS-COV-2 E) or an amino acid sequence with at least 90% sequence identity thereof; and
wherein the nucleic acid sequence has no sequence part that encodes an amino acid sequence having the function of a SARS-COV-2 amino acid sequence encoded by SEQ ID NO: 4(SARS-COV-2 M).

3. The nucleic acid sequence of claim 2, wherein the nucleic acid sequence comprises three primary nucleic acid sequence parts:
i) SEQ ID NO: 1 (SARS-COV-2 N) or an amino acid sequence with at least 90% sequence identity thereof;
ii) SEQ ID NO: 2 (SARS-COV-2 S) or an amino acid sequence with at least 90% sequence identity thereof; and
iii) SEQ ID NO: 3 (SARS-COV-2 E) or an amino acid sequence with at least 90% sequence identity thereof.

4. The nucleic acid sequence of claim 2, wherein
1.) the nucleic acid sequence comprises no nucleic acid sequence part encodes an amino acid sequence having the function of a SARS-COV-2 amino acid sequence encoded by ORF7 and ORF8; )
2.) the nucleic acid sequence comprises no nucleic acid sequence part encodes an amino acid sequence having the function of a SARS-COV-2 amino acid sequence encoded by ORF6 and ORF7ab; or)
3.) the nucleic acid sequence comprises no nucleic acid sequence part encodes an amino acid sequence having the function of a SARS-COV-2 amino acid sequence encoded by ORF6, ORF7ab and ORF8.

5. The nucleic acid sequence of claim 4, wherein the nucleic acid sequence comprises a primary nucleic acid sequence part encoding an amino acid sequence a) i), a secondary nucleic acid sequence part encoding an amino acid sequence having the function of a SARS-COV-2 amino acid sequence encoded by ORF3a and a sequence part of the nucleic acid sequence located between the primary nucleic acid sequence part encoding an amino acid sequence a) i) and the secondary nucleic acid sequence part encoding an amino acid sequence having the function of a SARS-COV-2 amino acid sequence encoded by ORF3a, wherein the sequence part comprises
I) SEQ ID NO: 35 or a sequence having at least 90% sequence identity to SEQ ID NO: 35;
II) SEQ ID NO: 36 or a sequence having at least 90% sequence identity to SEQ ID NO: 36; or
III) SEQ ID NO:37 or a sequence having at least 90% sequence identity to SEQ ID NO: 37.

6. A biologically produced nucleic acid sequence comprising two or three nucleic acid sequence parts encoding an amino acid sequence selected from the group consisting of:
i) SEQ ID NO: 1 (SARS-COV-2 N) or an amino acid sequence with at least 90% sequence identity thereof;
ii) SEQ ID NO: 2 (SARS-COV-2 S) or an amino acid sequence with at least 90% sequence identity thereof; and
iii) SEQ ID NO: 3 (SARS-COV-2 E) or an amino acid sequence with at least 90% sequence identity thereof; and
wherein the nucleic acid sequence has no sequence part that encodes an amino acid sequence having the function of a SARS-COV-2 amino acid sequence encoded by SEQ ID NO: 4 (SARS-COV-2 M), preferably wherein the nucleic acid sequence comprises a sequence as defined by SEQ ID NO: 33.

7. The nucleic acid sequence of claim 3, wherein the nucleic acid sequence comprises two primary nucleic acid sequence parts:
i) SEQ ID NO: 1 (SARS-COV-2 N) or an amino acid sequence with at least 90% sequence identity thereof; and
ii) SEQ ID NO: 2 (SARS-COV-2 S) or an amino acid sequence with at least 90% sequence identity thereof; and
wherein the nucleic acid sequence has no sequence part that encodes an amino acid sequence having the function of a SARS-COV-2 amino acid sequence encoded by SEQ ID NO: 4 (SARS-COV-2 M) and SEQ ID NO: 3 (SARS-COV-2 E), preferably wherein the nucleic acid sequence comprises a sequence as defined by SEQ ID NO: 34.

8. The nucleic acid sequence of claim 1, wherein for the secondary nucleic acid sequence part encoding an amino acid sequence having the function of a SARS-COV-2 amino acid sequence
i) ORF3a is a sequence defined by SEQ ID NO: 5;
ii) ORF6 is a sequence defined by SEQ ID NO: 6;
iii) ORF7a is a sequence defined by SEQ ID NO: 7; and/or
iv) ORF8 is a sequence defined by SEQ ID NO: 9.

9. The nucleic acid sequence of claim 1, wherein the nucleic acid sequence comprises three primary nucleic acid sequence parts.

10. The nucleic acid sequence of claim 1, wherein one of the secondary nucleic acid sequence parts encodes an amino acid sequence having the function of a SARS-COV-2 amino acid sequence encoded by ORF3a.

11. The nucleic acid sequence of claim 1, wherein the primary nucleic acid sequence parts and the secondary nucleic acid sequence parts are ordered in 5′ to 3′ direction in the following order:
1. SEQ ID NO: 2 (SARS-COV-2 S) or an amino acid sequence with at least 90% sequence identity thereof,
2. nucleic acid sequence part encoding an amino acid sequence having the function of a SARS-COV-2 amino acid sequence encoded by ORF3a;
3. SEQ ID NO: 3 (SARS-COV-2 E) or an amino acid sequence with at least 90% sequence identity thereof,
4. SEQ ID NO: 4 (SARS-COV-2 M) or an amino acid sequence with at least 90% sequence identity thereof,
5. nucleic acid sequence part encoding an amino acid sequence having the function of a SARS-COV-2 amino acid sequence encoded by ORF6,
6. nucleic acid sequence part encoding an amino acid sequence having the function of a SARS-COV-2 amino acid sequence encoded by ORF7a,
7. nucleic acid sequence part encoding an amino acid sequence encoding an amino acid sequence having the function of a SARS-COV-2 amino acid sequence encoded by ORF8,
8. SEQ ID NO: 1 (SARS-COV-2 N) or an amino acid sequence with at least 90% sequence identity thereof.

12. The nucleic acid sequence of claim 1, wherein the nucleic acid sequence comprises a nucleic acid sequence defined by the SEQ ID NO: 10 (SARS-COV-2 genome) or a sequence with at least 90% sequence identity thereof with a deletion and/or a dysfunctionality of:
a) the E gene, ORF6 gene, ORF7a gene and ORF8 gene; or
b) the E gene, ORF6 gene and ORF8 gene.

13. A vector comprising the nucleic acid sequence of claim 1.

14. (canceled)

15. The vector of claim 13, wherein the vector comprises
a) a sequence as defined by SEQ ID NO: 11 (biologically produced vector with ORF7a gene) or a sequence having 90% sequence identity thereof; or
b) a sequence as defined by SEQ ID NO: 12 (biologically produced vector without ORF7a gene) or a sequence having 90% sequence identity thereof.

16. A host cell comprising the nucleic acid sequence of claim 1.

17. The host cell of claim 16 additionally comprising at least one complementary SARS-COV-2 sequence thereof.

18. A method of production of a virus envelope and/or a fragment of a virus envelope and/or virus envelope protein comprising culturing the host cell of claim 16.

19. A kit comprising
I.) the nucleic acid sequence of claim 1; and
II.) at least one SARS-COV-2 sequence part complementary to the nucleic acid sequence comprised in (I.).

20. A virus envelope or a fragment of a virus envelope and/or virus envelope protein, wherein the virus envelope or the fragment of a virus envelope and/or the virus envelope protein
a) packages package-the at least one nucleic acid of claim 1; and
b) is are obtainable by gene expression using at least one nucleic acid of claim 1,

21. A pharmaceutical composition comprising
a) at least one nucleic acid according to claim 1, and
b) at least one amino acid sequence obtainable by gene expression using at least one nucleic acid of claim 1.

22. The pharmaceutical composition of claim 21, wherein the at least one amino acid sequence is the virus envelope or a fragment of a virus envelope and/or virus envelope protein of claim 20.

23. (canceled)

24. (canceled)

25. A method of preventing a SARS-COV-2 infection or at least one symptom thereof comprising administering an effective amount of the pharmaceutical composition according to claim 21 to a subject.
